(19) United States
(12) Patent Application Publication
(10) Pub. No.: US 2009/0123575 A1
(43) Pub. Date: May 14, 2009

(54) Blended Compositions for Treatment of Alzheimer's Disease and Other Amyloidoses

(76) Inventors: Thomas Lake, Snohomish, WA (US); Alan Snow, Lynnwood, WA (US)

Correspondence Address: PROTEOTECH, INC., 12040 115TH AVE NE, KIRKLAND, WA, 98034-6931, US

(21) Appl. No.: 12/249,115

(22) Filed: Oct. 10, 2008

Related U.S. Application Data
(60) Provisional application No. 60/987,307, filed on Nov. 12, 2007.

(51) Int. Cl. A61K 36/82; A61K 36/16; A61K 36/9066; A61K 36/87; C12N 5/00; A61K 131/00; A61P 25/00; A61P 25/28

(52) U.S. Cl. 424/729; 424/752; 424/756; 424/766; 435/375

(57) ABSTRACT

Methods for treating amyloid disease in a patient wherein the method comprises administration of a therapeutically effective amount of a composition comprising Uncaria tomentosa extract, Gingko Biloba, Green Tea Extract, Grape Seed Extract and/or Curcumin.

### TECHNICAL FIELD

The invention relates to blended compositions and methods for treating Alzheimer's Disease and other amyloidoses such as Parkinson's Disease; more particularly, it relates to blended compositions for therapeutic intervention in Alzheimer's and Parkinson's disease and other amyloidoses.

### BACKGROUND OF THE INVENTION

### Brain Amyloid Prevention and Memory/Recall Optimization

It is known that amyloid accumulates in the brains of people as they age. This amyloid is most commonly and most deleteriously in the form of what are known as amyloid plaques. In addition there are amyloid deposits in cerebral blood vessels. These accumulations form a brain amyloid burden that increases with age, so that age is a risk factor for Alzheimer's disease and other amyloidoses.

One of the most notable effects of increasing brain amyloid burden, and especially in Alzheimer's Disease, is the gradual deterioration of short term memory; that is, the ability to recall immediately those memories only recently stored.

Alzheimer's disease in general is characterized by the accumulation of a 39-43 amino acid peptide termed the beta-amyloid protein or Aβ, in a fibrillar form, existing as extracellular amyloid plaques and as amyloid within the walls of cerebral blood vessels. Fibrillar Aβ amyloid deposition in Alzheimer's disease is believed to be detrimental to the patient and eventually leads to toxicity and neuronal cell death, characteristic hallmarks of Alzheimer's disease. Accumulating evidence implicates amyloid as a major causative factor of Alzheimer's disease pathogenesis.

Parkinson's disease is also a neurodegenerative disorder, and it is pathologically characterized by the presence of intracytoplasmic Lewy bodies, the major components of which are filaments consisting of alpha-synuclein. Two dominant mutations in alpha-synuclein causing familial early onset Parkinson's disease have been described suggesting that Lewy bodies contribute mechanistically to the degeneration of neurons in Parkinson's disease. Alpha-synuclein fibril formation resembles that of Alzheimer's beta-amyloid protein (Aβ) fibrils. Parkinson's disease alpha-synuclein fibrils, like the Aβ fibrils of Alzheimer's disease, also consist of a predominant beta-pleated sheet structure.

A variety of other human diseases also demonstrate amyloid deposition and usually involve systemic organs (i.e. organs or tissues lying outside the central nervous system), with the amyloid accumulation leading to organ dysfunction or failure. In Alzheimer's disease and other “systemic” amyloid diseases, there are currently no cures or effective treatments, and the patient usually dies within 3 to 10 years from disease onset. For additional background in this area, the reader is referred to WIPO International publication number W098/51302 by the same inventors, the text of which is hereby incorporated by reference as if fully set forth herein.

Additional compounds or agents for therapeutic regimes to arrest or reverse amyloid formation, deposition, accumulation and/or persistence that occurs in Alzheimer's disease and other amyloidoses are still needed.

### DISCLOSURE OF THE INVENTION

An object of the present invention is to use the inner bark and/or roots from Uncaria tomentosa (also referred to as Una de Gato or Cat's claw) for the treatment/inhibition of amyloid formation, deposition, accumulation and/or persistence in Alzheimer's disease, type II diabetes and other amyloidoses, in conjunction with one or more of the additional blended ingredients disclosed below to achieve a beneficial synergistic therapeutic effect. Uncaria tomentosa or Cat's claw is also referred to as, but not limited to, Paraguayo, Garabato, Garbato casha, Tambor huasca, Una de gavilan, Hawk's claw, Nail of Cat, and Nail of Cat Schuler.

Another object of the present invention is to provide the use of Uncaria tomentosa with its blended compounds (regardless of commercial source and regardless of final form for consumption by humans, i.e. pills, tablets, caplets, soft and hard gelatin capsules, lozenges, sachets, cachets, vegicaps, liquid drops, elixirs, suspensions, emulsions, solutions, syrups, tea bags, aerosols (as a solid or in a liquid medium), suppositories, sterile injectable solutions, sterile packaged powders, bark bundles and/or bark powder) for inhibition of amyloid formation, deposition, accumulation, and/or persistence, regardless of its clinical setting.

These and such other objects of the invention as will become evident from the disclosure below are met by the invention disclosed herein.

Application of the invention to these needs is especially beneficial in that the invention is the only system that effectively provides for use of extracts from the inner bark and root parts of Uncaria tomentosa, together with hitherto unknown blended additional compounds, to benefit human patients with Alzheimer's disease and other amyloidoses due to the newly discovered ability of Uncaria tomentosa in combination with one or more of these blended ingredients, to effectively inhibit amyloid fibril formation, inhibit amyloid fibril growth, inhibit amyloid—proteoglycan interactions, amyloid—glycosaminoglycan interactions, and cause dissolution and/or disruption of preformed amyloid fibrils.

We have earlier discovered and disclosed a naturally occurring plant product, the inner bark and/or roots from the plant Uncaria tomentosa, or Cat's Claw, that we call PTI-00703, in WIPO International publication number W098/51302 entitled ‘Composition and Methods for Treating Alzheimer's Disease and other Amyloidoses’ dated Nov. 19, 1998. As disclosed therein, this plant compound alone has surprising efficacy in disrupting and/or dissolving amyloid deposits and other accumulations, and is believed to be a potent inhibitor of amyloid formation in Alzheimer's Disease, Type II Diabetes, and other amyloidoses. It is now also believed that formulations of PTI-00703 with other plant compounds, herbals, minerals, and/or vitamins as disclosed herein have surprising and hitherto unsuspected supplementary efficacy in treating the various amyloidoses addressed by our earlier disclosure for PTI-00703 by itself.

### Alzheimer's Disease

PTI-00703 is advantageously blended with one or more of the following ingredients for treatment of amyloidoses such as Alzheimer's and Parkinson's disease, and for improved brain cognition, memory/recall optimization and the like.
1) Gingko Biloba, an herb that enhances memory;
2) Ginseng, plants in the genus Panax use for promoting well-being and energy;
3) Gotu Kola, an herb that increases energy, endurance, memory and mental stamina;
4) Echinacea, an herb with potent anti-oxidant activity;
5) Vitamin E, an anti-oxidant;
6) Selenium, an anti-oxidant;
7) Niacin, or nicotinate, a B vitamin that helps with cell metabolism and cell energy;
8) Folic acid, part of B-complex that helps with cardiovascular function and circulatory health;
9) Vitamin B12, or cobalamin, important in cell metabolism and cell energy;
10) Choline, precursor to acetylcholine that is important in essential brain neurotransmission.

Optimal formulations of PTI-00703 contain one or more of these ingredients. It is expected that increasing PTI-00703 dosage should occur with older people (i.e. different regimes for people age 20-40 years old; 40-60 years old and >60 years old) so as best to accommodate the risk factor described above.

The invention relates to the use of mixed compositions (see Table 1) including one or more of the following: grape seed extract, green tea extract, gingko biloba, curcumin, and Uncaria tomentosa extract PTI-00703 referred to as ‘703’ for the therapeutic intervention of Alzheimer's disease and other amyloidoses such as Parkinson's and Lewy body diseases. Use of mixed compositions of grape seed extract green tea extract, gingko biloba, curcumin, and/or PTI-00703, contained within different commercial preparations show unexpected effects on the inhibition of both Aβ 1-42 and α-synuclein fibrillogenesis.

Parkinson's disease is another amyloidosis characterized by the accumulation of fibrils in the brains of patients with this disease (which are Congo red and Thioflavin T positive, and which contain predominant beta-pleated sheet secondary structure). Agents or compounds found to inhibit Alzheimer's disease Aβ amyloid fibril formation have also proven to be effective in the inhibition of α-synuclein fibril formation. These agents or compounds will therefore also serve as therapeutics for Parkinson's and Lewy body disease, in addition to having efficacy as a therapeutic for Alzheimer's disease and other amyloid disorders.

These and other features and advantages of the present invention will become more fully apparent when the following detailed description of the invention is read in conjunction with the accompanying figures.

A preferred pharmacological agent preferably has a therapeutically effective amount of Uncaria tomentosa in a dosage in the range of from about 10 to 1,000 mg/kg of body weight of the patient, and more preferably in the range of from about 10 to 100 mg/kg of body weight of the patient.

The compositions preferably have a therapeutically effective amount of the mixed composition of grape seed extract, green tea extract, gingko biloba, curcumin, and/or PTI-00703, in a dosage in the range of from about 0.1 to about 500 mg/kg of body weight of the patient, and more preferably in the range from about 1.0 to about 100 mg/kg of body weight of the patient.

Preferred pharmaceutical agents have a weight percentage of plant extract in the agent is in the range of from about 70% to about 95%, and may also have a pharmaceutically acceptable carrier, diluent or excipient. The pharmaceutical agent preferably has an amyloid inhibitory activity or efficacy greater than 50%.

The plant matter is preferably comprised of commercially obtained pills, tablets, caplets, soft and hard gelatin capsules, lozenges, sachets, cachets, vegicaps, liquid drops, elixers, suspensions, emulsions, solutions, syrups, tea bags, aerosols (as a solid or in a liquid medium), suppositories, sterile injectable solutions, sterile packaged powders, bark bundles and/or bark powder, which contain Uncaria tomentosa, extracts or derivatives thereof, and may be taken from commercially available gelatin-coated capsules which contain dried powder of Uncaria tomentosa, extracts or derivatives thereof.

A method is also disclosed for treating an amyloid disease in a patient, comprising the step of administering to the patient a therapeutically effective amount of plant matter from a plant of the genus Uncaria, species tomentosa, in combination with one or more of the additional blend ingredients disclosed above. The plant matter is preferably administered orally or by aerosol spray or in a parenterally injectable or infusible form.

### EXAMPLES

20-40 Years of Age

400 mg of PTI-00703 plus 50 mg of a mixture of all ten of the above listed ingredients (i.e. 5 mg per ingredient)=450 mg total, with a weight ratio of 8:1 PTI -00703 to total other ingredients. Mixture may be taken orally (or the like) in gel caps 3× day.

40-60 Years of Age

600 mg of PTI-00703 plus 75 mg of a mixture of all ten of the above ingredients (i.e. 7.5 mg per ingredient)=675 mg total, with a weight ratio of 8:1 PTI-00703 to total other ingredients. Mixture may be taken orally (or the like) in gel caps 3× day.

>60 Years of Age, or Diagnosed with Alzheimer's Disease

800 mg of PTI-00703 plus 100 mg of a mixture of all ten of the above ingredients (i.e. 10 mg per ingredient)=900 mg total, with a weight ratio of 8:1 PTI-00703 to total other ingredients. Mixture may be taken orally (or the like) in gel caps 4× day.

### Pancreatic Amyloid Diabetes Prevention and Beta Cell Optimization

It is also known that amyloid accumulates in the pancreas of 90% of all patients with Type II Diabetes, and that this amyloid accumulation contributes to pancreatic organ dysfunction. For one thing, amyloid is toxic to beta cells which normally produce insulin, and it has been observed that patients with such amyloid accumulation are at risk of becoming insulin dependent (i.e. Type I Diabetes). For further background on pancreatic amyloidosis, the reader is referred to the inventors earlier WIPO International publication number WO98/51302.

PTI-00703 is advantageously blended with one or more of the following ingredients for type II diabetes amyloidosis and beta cell optimization and the like.
1) Bilberry, an herb with circulation enhancing properties;
2) Dong Quai, an herb that helps maintain immune function, blood pressure, and good circulation;
3) Aloe Vera, an herb that helps promote healthy lower intestine function;
4) Chromium Polynicotinate, an organic complex of chromium and picohnic acid that works to metabolize the body's fat; and cholesterol;
5) Selenium, an anti-oxidant that neutralizes free radicals that can otherwise damage cells
6) Vitamin B12, or cobalamin, important in cell metabolism and cellular level energy production;
7) Folic acid, a part of the B-complex that helps with cardiovascular function and circulatory system health;
8) Biotin, a part of the B-complex that also promotes circulatory health;
9) Thiamine HCl, or vitamin B1, helps maintain blood sugar levels by increasing metabolic efficiency and helps maintain cardiovascular function.

Optimal formulations of PTI-00703 contain one or more of these ingredients. It is expected that increasing PTI-00703 dosage should occur with older people (i.e. different regimes for people age 20-40 years old and >40 years old and for people who have diagnosed type II diabetes) so as best to accommodate the risk factor described above.

### EXAMPLES

20-40 Years of Age

300 mg of PTI-00703 plus 45 mg of a mixture of all nine of the above ingredients (i.e. 5 mg per ingredient)=345 mg total, with a weight ratio of 8:1 PTI-00703 to total other ingredients. Mixture may be taken orally (or the like) in gel caps 3× day.

>40 Years of Age

500 mg of PTI-00703 plus G3 mg of a mixture of all nine of the above ingredients (i.e. 7 mg per ingredient)=5G3 mg total, with a weight ratio of 8:1 PTI-00703 to total other ingredients. Mixture may be taken orally (or the like) in gel caps 3× day.

Diagnosed Type II Diabetes

700 mg of PTI-00703 plus 90 mg of a mixture of all ten of the above ingredients (i.e. 10 mg per ingredient)=790 mg total, with a weight ratio of 8:1 PTI-00703 to total other ingredients. Mixture may be taken orally (or the like) in gel caps 4× day.

### BRIEF DESCRIPTION OF THE DRAWINGS

FIG. 1 is a graph of a sequence of a Thioflavin T fluorometry assay of one of the combinations of the invention.

FIG. 2 is a graph of a Thioflavin T fluorometry assay utilized to assess effectiveness of one of the combinations of the invention for its inhibition of Alzheimer's Aβ-Aβ interactions.

FIG. 3 is a graph of a Thioflavin T fluorometry assay utilized to assess effectiveness of one of the combinations of the invention for its inhibition of Alzheimer's Aβ-proteoglycan/glycosaminoglycan (PG/GAG) interactions.

FIG. 4 is a graph of a Thioflavin T fluorometry assay utilized to assess effectiveness of one of the combinations of the invention in dissolution of pre-formed Alzheimer's Aβ 1-40 amyloid fibrils in a dose-dependent manner.

FIG. 5 is a graph of a Thioflavin T fluorometry assay utilized to assess effectiveness of one of the combinations of the invention in dissolution of pre-formed Alzheimer's Aβ 1-42 amyloid fibrils in a dose-dependent manner.

FIG. 6 is a graph showing % inhibition of disassembly/disruption of Aβ 1-42 amyloid fibrils by the compositions listed in Table 1 measured by Thioflavin T fluorometry assay at 1:0.1.

FIG. 7 is a graph showing % inhibition of disaggregation of Aβ 1-42 amyloid fibrils by the compositions listed in Table 1 measured using Congo Red assay at 1:1.

FIG. 8 is a graph showing % inhibition of disassembly/disruption of α-synuclein fibrils by the compositions listed in Table 1 measured by Thioflavin T fluorometry assay at 1:0.1.

FIG. 9 is a graph showing % inhibition of disaggregation of α-synuclein fibrils by the compositions listed in Table I measured using Congo Red assay at 1:1.

### DETAILED DESCRIPTION OF THE INVENTION

“Mammal” includes both humans and non-human mammals, such as companion animals (cats, dogs, and the like), laboratory animals (such as mice, rats, guinea pigs, and the like) and farm animals (cattle, horses, sheep, goats, swine, and the like).

“Treating” or “treatment” of a disease includes preventing the disease from occurring in a mammal that may be predisposed to the disease but does not yet experience or exhibit symptoms of the disease (prophylactic treatment), inhibiting the disease (slowing or arresting its development), providing relief from the symptoms or side-effects of the disease (including palliative treatment), and relieving the disease (causing regression of the disease), such as by disruption of pre-formed amyloid or synuclein fibrils. One such preventive treatment may be use of the disclosed compounds for the treatment of Mild Cognitive impairment (MCI).

A “therapeutically effective amount” in general means the amount that, when administered to a subject or animal for treating a disease, is sufficient to affect the desired degree of treatment for the disease. A “therapeutically effective amount” or a “therapeutically effective dosage” preferably inhibits, reduces, disrupts, disassembles amyloid or synuclein fibril formation, deposition, accumulation and/or persistence, or treats a disease associated with these conditions, such as an amyloid disease or a synucleinopathy, by at least 20%, more preferably by at least 40%, even more preferably by at least 60%, and still more preferably by at least 80%, relative to an untreated subject. Effective amounts of a compound of this invention or composition thereof for treatment of a mammalian subject are about 0.1 to about 1000 mg/Kg of body weight of the subject/day, such as from about 1 to about 100 mg/Kg/day, especially from about 10 to about 100 mg/Kg/day. A broad range of disclosed composition dosages are believed to be both safe and effective.

“Fibrillogenesis” refers to the formation, deposition, accumulation and/or persistence of amyloid fibrils, filaments, inclusions, deposits, as well as synuclein (usually involving α-synuclein) and/or NAC fibrils, filaments, inclusions, deposits or the like.

“Inhibition of fibrillogenesis” refers to the inhibition of formation, deposition, accumulation and/or persistence of such amyloid fibrils or synuclein fibril-like deposits.

“Disruption of fibrils or fibrillogenesis” refers to the disruption of pre-formed amyloid or synuclein fibrils that usually exist in a pre-dominant β-pleated sheet secondary structure. Such disruption by compounds of the invention may involve marked reduction or disassembly of amyloid or synuclein fibrils as assessed by various methods such as circular dichroism spectroscopy, Thioflavin T fluorometry, Congo red binding, SDS-PAGE/Western blotting, as demonstrated by the Examples presented in this application.

“Amyloid diseases” or “amyloidoses” suitable for treatment with the compounds of this invention are diseases associated with the formation, deposition, accumulation, or persistence of amyloid fibrils, especially the fibrils of an amyloid protein selected from the group consisting of Aβ amyloid, AA amyloid, AL amyloid, IAPP amyloid, PrP amyloid, α₂-microglobulin amyloid, transthyretin, prealbumin, and procalcitonin, especially Aβ amyloid and IAPP amyloid. Suitable such diseases include Alzheimer's disease, Down's syndrome, dementia pugilistica, multiple system atrophy, inclusion body myositosis, hereditary cerebral hemorrhage with amyloidosis of the Dutch type, Nieman-Pick disease type C, cerebral β-amyloid angiopathy, dementia associated with cortical basal degeneration, the amyloidosis of type 2 diabetes, the amyloidosis of chronic inflammation, the amyloidosis of malignancy and Familial Mediterranean Fever, the amyloidosis of multiple myeloma and B-cell dyscrasias, the amyloidosis of the prion diseases, Creutzfeldt-Jakob disease, Gerstmann-Straussler syndrome, kuru, scrapie, the amyloidosis associated with carpal tunnel syndrome, senile cardiac amyloidosis, familial amyloidotic polyneuropathy, and the amyloidosis associated with endocrine tumors, especially Alzheimer's disease and type 2 diabetes.

“Synuclein diseases” or “synucleinopathies” suitable for treatment with the compounds of this invention are diseases associated with the formation, deposition, accumulation, or persistence of synuclein fibrils, especially α-synuclein fibrils. Suitable such diseases include Parkinson's disease, familial Parkinson's disease, Lewy body disease, the Lewy body variant of Alzheimer's disease, dementia with Lewy bodies, multiple system atrophy, and the Parkinsonism-dementia complex of Guam.

### Amyloid and Amyloidosis

Amyloid is a generic term referring to a group of diverse, but specific extracellular protein deposits which all have common morphological properties, staining characteristics, and x-ray diffraction spectra, further details and information as to which, and as to amyloid as a therapeutic target for Alzheimer's Disease, the reader is referred to the inventors WIPO International publication number W098/51302.

Uncaria tomentosa

The plant Uncaria tomentosa, also known as “Uña de Cato” (in Spanish) or “Cat's claw” (in English) refers to a woody vine which grows within the Peruvian Amazon rain forest. For additional and further information and background on Uncaria tomentosa, the reader is also referred to the inventors' WIPO International publication number W098/51302.

Although some health care providers have suggested that Uncaria tomentosa may be used to treat a variety of ailments, nowhere has there been any use, or suggestion of use, of this compound for the treatment of amyloid formation, deposition, accumulation and/or persistence, such as that which occurs in the amyloidoses, including Alzheimer's disease, and nowhere is it suggested that certain other compounds might have synergistic or supplemental efficacy in combination with Uncaria tomentosa in treating amyloidoses. The present invention clearly demonstrates the effectiveness of Uncaria tomentosa and its combinations for 1) inhibition of Alzheimer's Aβ amyloid fibril formation (important for patients in early to mid-stage Alzheimer's disease), 2) inhibition of Alzheimer's amyloid fibril growth (important for patients in early to mid-stage Alzheimer's disease), 3) inhibition of Alzheimer's amyloid-PG/GAG interactions (important for patients in all stages of Alzheimer's disease) and 4) causing the dissolution/disruption of preformed Alzheimer's disease amyloid fibrils. In addition, the present invention and its combinations are effective in causing the dissolution of islet amyloid fibrils (i.e. amylin) and therefore serves as an effective treatment for −90% of type II diabetic patients who have islet amyloid accumulation in the pancreas.

### Methodologies Employed for In Vitro Testing

### Generation of “Water Extracts” for In Vitro Testing

For the procedure to generate water extracts of PTI-00703, 500 mg PTI-00703 were extracted with 3 ml of distilled water (Baxter) and placed in microcentrifuge tubes. The microcentrifuge tube contents were then vortexed by hand for 3-4 minutes, and then allowed to stand for 1-2 minutes. The samples were then centrifuged on a microcentrifuge (Eppendorf, model 5415 for 30 minutes at 14,000×g (at room temperature). Following centrifugation, the supernatants were collected and designated as the “water extracts” used for testing as described below.

### EXAMPLES

The following examples are put forth so as to provide those with ordinary skill in the art with the disclosure and description of the identification and use of commercially available Uncaria tomentosa and disclosed blend ingredients to inhibit amyloid fibril formation, inhibit amyloid fibril growth, inhibit amyloid-PG/GAG interactions, and cause dissolution/disruption of preformed amyloid fibrils. However, it should not be construed that the invention is limited to these specific examples.

The PTI-00703, is in the form of Cat's Claw Bark Powder, and the blend testing illustrated below is of 350 mg of Cat's Claw Bark Powder and 40 mg of Gingko biloba powder extract, or Gingko biloba leaf extract containing standardized 24% gingkoflavoglycosides and 6% terpene lactones; total 390 mg per test capsule.

### Study 1: Testing to Assess Effects on Alzheimer's Disease Amyloid Fibril Formation

A previously described method of measuring amyloid fibril formation utilizing Thioflavin T fluorometry (H Naiki et al, Lab. Invest. 65: 104-110, 1991; H Levine III, Protein Sci. 2:404-410, 1993; H Levine III, Amyloid: Int. J. Exp. Clin. Invest. 2: 1-6, 1995; H Naiki and K. Nakakuki, Lab. Invest. 74: 374-383, 1996) was employed initially to identify whether PTI-00703 and PTI-00703 blended with Gingko biloba were capable of inhibiting Alzheimer's Aβ 1-40 amyloid fibril formation. Using this sensitive assay, any decreases or increases in fluorescence was previously shown to correlate with a decrease or increase in the amount of amyloid fibrils (H Naiki et al, Lab. Invest. 65: 104-110, 1991; H Levine III, Protein Sci. 2: 404-410, 1993; H Levine III, Amyloid: Int. J. Exp. Clin. Invest. 2: 1-6, 1995; H Naiki and K. Nakakuki, Lab. Invest. 74: 374-383, 1996), allowing one to determine the identity and extent of potential inhibitors and/or enhancers of amyloid fibril formation.

In one study, the dose-dependent effects of PTI-00703 and its Gingko biloba blend on Alzheimer's Aβ (1-40) fibril formation was assessed by Thioflavin T fluorometry. Thioflavin T is known to bind fibrillar amyloid proteins and an increase in fluorescence correlates with an increase in amyloid fibril formation, whereas a decrease in fluorescence correlates with a decrease in amyloid fibril formation. The Alzheimer's Aβ protein (1-40) when incubated at 37° C. tends to spontaneously form amyloid fibrils which increase in quantity over time. In this study, we tested for ability to inhibit the Alzheimer's amyloid Aβ protein from forming fibrils over a 1 week period. For these studies, 300 μl of 25 μM Aβ (1-40) (Bachem Inc., Torrance, Calif., USA; Lot: #T20824) in 150 mM TRIS, 10 mM NaCl, pH 7.0 (TBS) was incubated in microcentrifuge tubes at 37° C. for 1 week (in triplicate), either alone, or in the presence of increasing concentrations (i.e. 0.01 μl, 0.1 μl, 0.5, ul and 1.0 μl) of a water extract (described below) of PTT-00703 and PTI-00703 with Gingko biloba (obtained as described above).

To assess the dose-dependent effects of these substances on Aβ (1-40) fibril formation, 50 μl aliquots were taken from each tube (as described above) for analysis at 1 hr, 1 day, 3 days, and 1 week. For each determination described above, following each incubation period, 50 μl of Aβ ± increasing concentrations of a water extract were added to 1.2 ml of 100 cM Thioflavin T (Sigma Chemical Co., St. Louis, Mo.) in 50 mM NaP04 (pH 6.0). Studies indicated that increasing concentrations of fibrillized Aβ gave a proportional increase in fluorescence in the presence of 100 uM Thioflavin T, ruling out the presence of any disproportionate inner filter effects in these studies.

Fluorescence emission at 482 nm was measured on a Turner instrument-model 450 fluorometer at an excitation wavelength of 450 nm. For each determination, the fluorometer was calibrated by zeroing in the presence of the Thioflavin T reagent alone, and by setting the 50 ng/ml riboflavin (Sigma Chemical Co., St. Louis, Mo.) in the Thioflavin T reagent to 1800 fluorescence units. All fluorescence determinations were based on these references and any fluorescence given off by any of the compounds tested in the presence of the Thioflavin T reagent was always subtracted from all pertinent readings.

For all fibrillogenesis studies utilizing Thioflavin T fluorometry, as disclosed herein, comparisons of amylold protein in the presence or absence of test compounds were based on paired Student's t tests with data shown as mean±standard deviation. Significance was reported at the 95% (p<0.05) and 99% (p<0.01) confidence levels.

### Study 2 Testing to Assess Effects on Alzheimer's Disease Amyloid Fibril Growth

In Alzheimer's disease and other amyloidoses, amyloid fibril growth is believed to involve amyloid protein self-interactions (i.e. AB-AB interactions). Any potential effective therapeutic agent for amyloid deposition, accumulation and/or persistence should also be capable of causing an inhibition of amyloid protein self-interactions. This is important for preventing any new amyloid fibril formation when treating Alzheimer's disease patients at early stages of the disease. ELISA methodologies (i.e. solid phase binding assays) were therefore used to identify compounds which were capable of inhibiting AB-AB interactions (i.e. Alzheimer's amyloid fibril growth).

Aβ (1-40) was first labeled with biotin according to the following protocol. 1 mg of Aβ (1-40) (Bachem Inc., Torrance, Calif., USA; Lot #WL934) was dissolved in 2001 of PBS (pH 8.0) and incubated for 1 week at 37° C. The fibrillar Aβ solution was then added to 0.2 mg of a biotinylation agent (sulfosuccinimidyl-G-(biotinamido)hexanoate) (sulfo-NHS LC-Biotin) and incubated for 45 minutes at room temperature (according to the manufacturer's protocol; Pierce). To remove excess sulfo-NHS-LC-Biotin not incorporated into Aβ, 25, al of 3M sodium acetate and 1 ml of ethanol were added to the solution, vortexed and then centrifuged at 14,000×g for 20 minutes. The supernatant was then discarded and the pellet was resuspended in 2001 of distilled water, and reprecipitated with ethanol containing 2.5% of 3M sodium acetate. The cenifilugation steps (described above) were then repeated. The pellet; which contained fibrillized Aβ which was biotinylated (at the non self interacting region of Aβ was then resuspended in 1 ml of distilled deionized water. The amount of biotin incorporated was then determined using the HABA (2-(4′-hydroxyazo benzene) benzoic acid) method (according to the manufacturer's protocol; Pierce).

Two (2) μg of unlabeled Aβ in 40 μl of Tris-buffered saline containing 100 mM Tris-HCl, 50 mM NaCl, 3 mM NaN3, pH 7.0 (TBS) was allowed to bind overnight at 4° C. to microtiter wells (Nunc plates, Maxisorb). The next day all of the microtiter wells were blocked for 2 hours by incubating with 300 ml of TBS with 0.05% Tween-20 (TTBS) plus 2% bovine serum albumin (BSA) (obtained from the Sigma Chemical Company, St. Louis, Mo., USA). Then, 100 ul of 12.5 μM biotinylated Aβ 1-40 in TTBS, in the presence or absence of 1 μl of water extracts (described above) were placed in wells (in triplicate) containing substrate bound unlabeled Aβ or blank, and allowed to bind overnight at 4° C. The next day, the wells were rinsed 3 times with TTBS, and then probed for 2 hours with 100, ul of streptavidin peroxidase or anti-biotinperoxidase (1:500 dilution of a 2 μg/ml solution) (Sigma Chemical Co., St. Louis, Mo.) in TTBS containing 0.1% BSA. The wells were then rinsed 3 times with TTBS and 100 μl of a substrate solution (OPD-Sigma Fast from Sigma Chemical Co., St. Louis, Mo.) was added to each well and allowed to develop for 5 minutes or until a significant color change was observed. The reaction was stopped with 50 ul of 4N H2S0₄ and read on a Model 450 microplate reader (Biorad, Hercules, Calif., USA) at 490 nm.

### Study 3: Testing to Assess Effects on Alzheimer's Disease All Glycosaminoglycan Interactions

One study was implemented to determine whether the test compounds were effective inhibitors of Aβ-proteoglyean/glycosaminoglyean (PG/GAG interactions. Since PGs/GAGs have been found to accumulate in amyloid deposits and are believed to prevent the body's natural ability to remove unwanted “amyloid” (reviewed in Snow and Wight, Neurobiology Aging 10:481-497, 1989)), an inhibitor of Aβ-PG/GAG interactions is a desirable additional target for an amyloid therapeutic. In this study a solid phase binding immunoassay was utilized to determine whether the test compounds were effective inhibitors of Aβ-PG/GAG interactions.

Twelve (12) ug of perlecan glycosaminoglycans (isolated from the Engelbreth-Holm-Swarm sarcoma as previously described (Castillo et al, J. Neurochemistry 69:2452-2465, 1997) in 40 μl of Tris-buffered saline containing 100 mM Tris-HCl, 50 mM NaCl, 3 mM NaN₃, pH 7.0 (TBS) was allowed to bind overnight at 4° C. to microtiter wells (Nunc plates, Maxisorb). The next day all of the microtiter wells were blocked for 2 hours by incubating with 300 ul of TBS with 0.05% Tween-20 (TTBS) plus 1% bovine serum albumin (BSA). 100 μl of Aβ 1-40 (12.5 μl) (Bachem Inc., Torrance, Calif., USA; Lot ##T20824)) in TTBS containing 1% albumin in the presence or absence of 1 ul of a water extract of the test compound, PTI-00703+Gingko biloba, were placed in wells (in triplicate) containing substrate bound perlecan GAGs or blank, and allowed to bind overnight at 4° C. The next day, the wells were rinsed 3 times with TTBS, and then probed for 2 hours with 100 μl of biotinylated anti-4G8 and anti-6E10 (Senetek, Maryland Heights, Missouri) diluted 1:2000 with TTBS. Bound antibodies were then probed with 100, ul of streptavidin peroxidase or anti-biotinperoxidase (1:500 dilution of a 2 ug/mil solution; Sigma Chemical Co., St. Louis, Mo.) in TTBS for 1 hour. The wells were then rinsed 3 times with TTBS and 100 ul of a substrate solution (OPD-Sigma Fast from Sigma Chemical Co., St. Louis, Mo.) was added to each well and allowed to develop for 5 minutes or until a significant color change was observed. The reaction was stopped with 50 ul of 4N H₂SO₄ and read on a Model 450 microplate reader (Biorad, Hercules, Calif., USA) at 490 nm.

### Study 4: Testing to Assess Dose-Dependent Effects on Causing a Dissolution/Disruption of Pre-Formed Alzheimer's Disease Amyloid 1-40 Fibrils

One study was implemented to determine whether extracts of the test compounds were capable of causing a “dissolution” or “disruption” of pre-formed Alzheime's disease amyloid fibrils. This type of activity would be important for any potential anti-amyloid drug which can be used in patients who already have substantial amyloid deposition in organs and/or tissues. For example, Alzheimer's disease patients in mid-to late stage disease have abundant amyloid deposits in their brains as part of both neuritic plaques and cerebrovascular amyloid deposits. A natural therapeutic agent capable of causing dissolution of pre-existing amyloid would be advantageous for use in these patients who are at latter stages of the disease process.

For this study, 1 mg of Aβ (1-40) (Bacllem Inc., Torrance, Calif., USA; Lot #T20824) was dissolved in 1.0 ml of double distilled water (1 mg/ml solution) and then incubated at 37° C. for 1 week to cause abundant Alzheimer's amyloid fibril formation. 25 uM of fibrillized Aβ was then incubated in triplicate for 2 hours at; 37° C. in a total final volume of 60 μl TBS, in the absence or presence of increasing concentrations (i.e. 0.01 μl, 0.1 μl, 0.5 μl and 1.0 μl) of test compound water extracts. Following a 2 hour incubation, 50 ul aliquot's were added to 1.2 ml of 100 uM Thioflavin T (Sigma Chemical Co., St. Louis, Mo.) in 50 nM NaP0₄ (pH 6.0) for fluorometry readings as described in experiment 1 described above.

### Study 5: Testing to Assess Dose-Dependent Effects on Causing a Dissolution/Disruption of Pre-formed Alzheimer's Disease Amyloid 1-42 Fibrils

The amyloid fibrils of Alzheimer's disease primarily consist of Aβ in a form containing residues 1-40 or 1-42. The longer variant of Aβ contains two hydrophobic residues which cause substantial fibril formation almost immediately (Castillo et al, J. Neurochem. 69: 2452-2465, 1997). Aβ 1-42 is also believed to be the predominant form of Aβ existing in Alzheimer's amyloid plaques, whereas Aβ 1-40 is believed to be the predominant form of Aβ existing in Alzheimer's cerebrovascular amyloid deposits (Tamaoka et al, Br. Res. 679: 151-156, 1995; Biochem. Biophys. Res. Comm. 205: 834-842, 1994). The next study was therefore implemented to determine whether the test compound also causes dissolution/disruption of pre-formed Aβ (1-42) amyloid fibrils and whether this effect was long-lasting.

For this study, the method of Thioflavin T fluorometry as described in Study 1 was used. Briefly, 60 ul of 25 uM of Aβ (1-42) (Bachem Inc, Torrance, Calif., USA; Lot#51G817) in TBS (pH 7.0) either alone, or containing increasing amounts (i.e. 0.01 μl, 0.1 μl, 0.5 μl and 1.0 μl) of test compound water extracts were incubated in microcentrifuge tubes at 37° C. for 48 hours (in triplicate).

### Results

### Study 1: Dose-Dependent Inhibition of Alzheimer's Disease Amyloid Fibril Formation

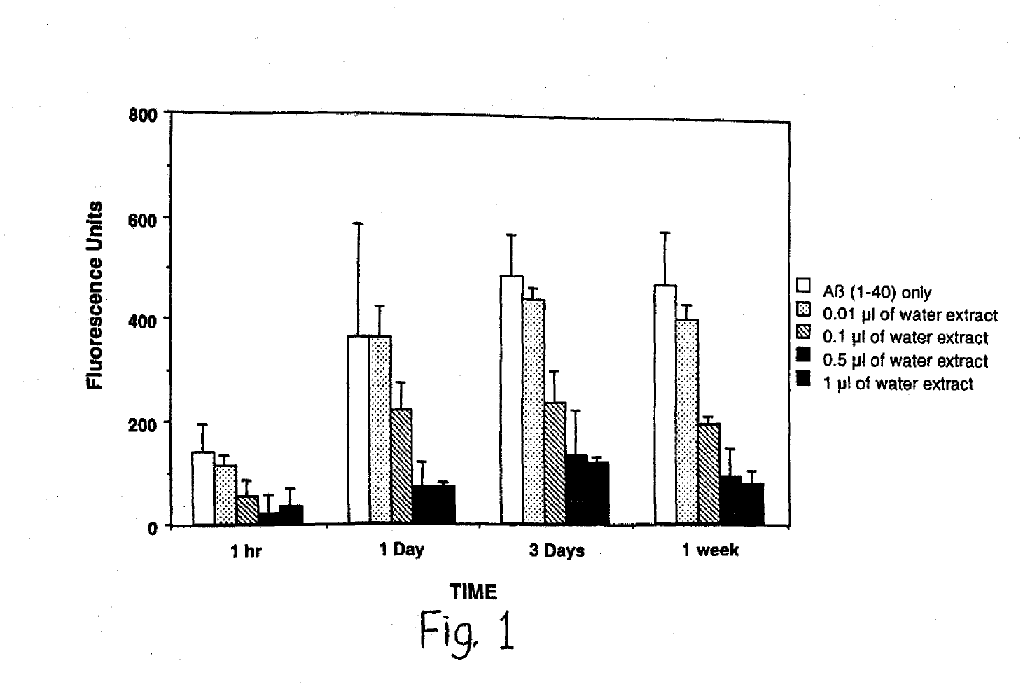

As shown in FIG. 1, the effects of various amounts (i.e. 0.01 μl, 0.1 μl, 0.5 μl and 1.0 μl) of PTI-00703 and Gingko biloba test compound on Alzheimer's Aβ (1-40) amyloid fibril formation was evaluated over a 1-week incubation period. Following a freeze-drying experiment to determine the weight of each of the water extracts (at each of the dilutions used), the following data for test compound water extracts was generated: 1 ul of water extract=23.0 ug of compound; 0.5 μl of water extract=1.5 μg; 0.1 ul of water extract=2.3 μg; 0.01 μl of water extract=0.23 μg.

In this study, freshly suspended Aβ (1-40) alone, following a 1-hour incubation at 37° C., demonstrated an initial fluorescence of 142±53 fluorescence units. During the 1-week incubation period, there was a gradual increase in the fluorescence of Aβ (1-40) alone, increasing 3.4-fold from 1 hour to 3 days, with a peak fluorescence of 487±82 fluorescence units observed at 3 days (FIG. 1). A significant inhibition (p<0.05) of Aβ 1-40 amyloid fibril formation by 1.0 ul of test compound was detected as early as I hour of incubation. Significant dose-dependent inhibition by increasing concentrations of the test compound on Aβ 1-40 amyloid fibril formation was observed at all time points including 1 hour, 1 day, 3 days and 1 week. At 1 hour, 0.5 μl (i.e. 11.5 ug) and 1.0 ul (i.e. 23.0 ug) of water extract inhibited Aβ 1-40 amyloid fibril formation by 68% and 77%, respectively. At 1 day, 0.5 ul (i.e. 11.5 ug) and 1.0 ul (i.e. 23.0 μg) of a water extract inhibited Aβ 1-40 amyloid fibril formation by 62% and 79%, respectively. At 1 week, increasing concentrations of test compound inhibited Aβ 1-40 fibril formation in a dose-dependent manner, such that 0.1 ul (i.e. 2.3 ug), 0.5 ul (i.e. 11.5 μg) and 1.0 μl (i.e. 23.0 μg) of water extract inhibited Aβ 1-40 amyloid fibril formation by 39%, 76%, and 86%, respectively. This initial data indicated that the test compound, PTI-00703+Gingko biloba, was a potent inhibitor of Alzheimer's amyloid fibril formation and exerted its effects in a dose-dependent manner.

### Study 2: Potent Inhibitor of Alzheimer's Disease Amyloid Fibril Growth

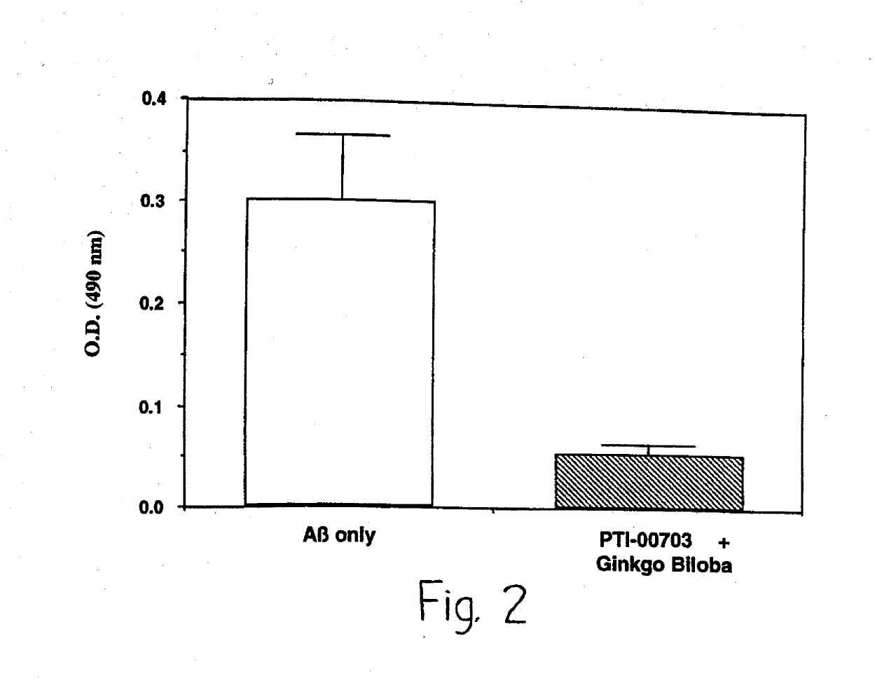

As shown in FIG. 2, test compound water extract was extremely effective in causing a significant reduction in Aβ-Aβ interactions. The extract caused a significant (p<0.01) 82% inhibition of Aβ-Aβ interactions. This data demonstrated that the test compound was a potent inhibitor of Aβ-Aβ interactions, indicative of inhibition of amyloid fibril growth.

### Study 3: Inhibition of Alzheimer's Beta-Amyloid Protein-Glycosaminoglycan Interactions

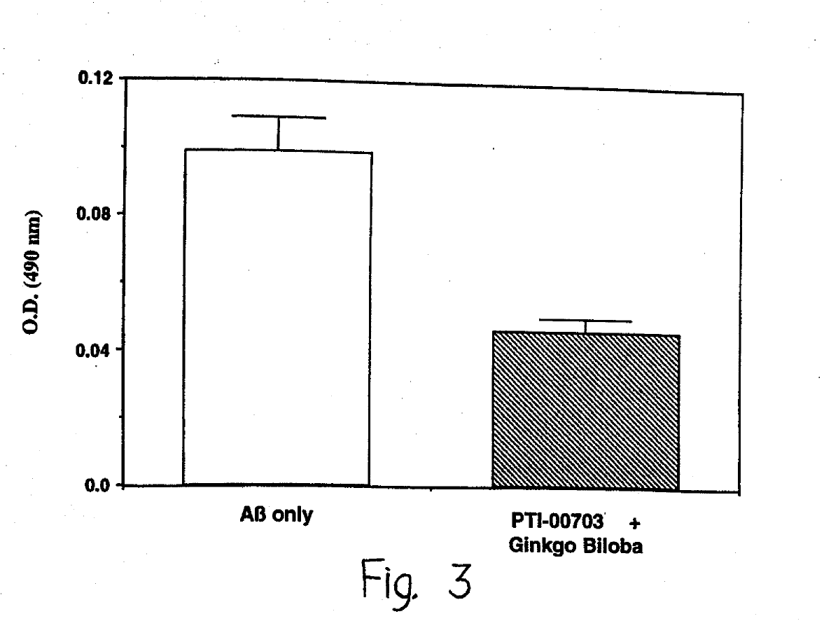

As shown in FIG. 3, the test compound significantly (p<0.01) inhibited Aβ-perlecan GAG interaction by 54%. This data demonstrated that it was also an inhibitor of beta-amyloid protein-PG/GAG interactions.

### Study 4: Dose-Dependent Disruption of Pre-Formed Alzheimer's Aβ (1-40) Amyloid Fibrils

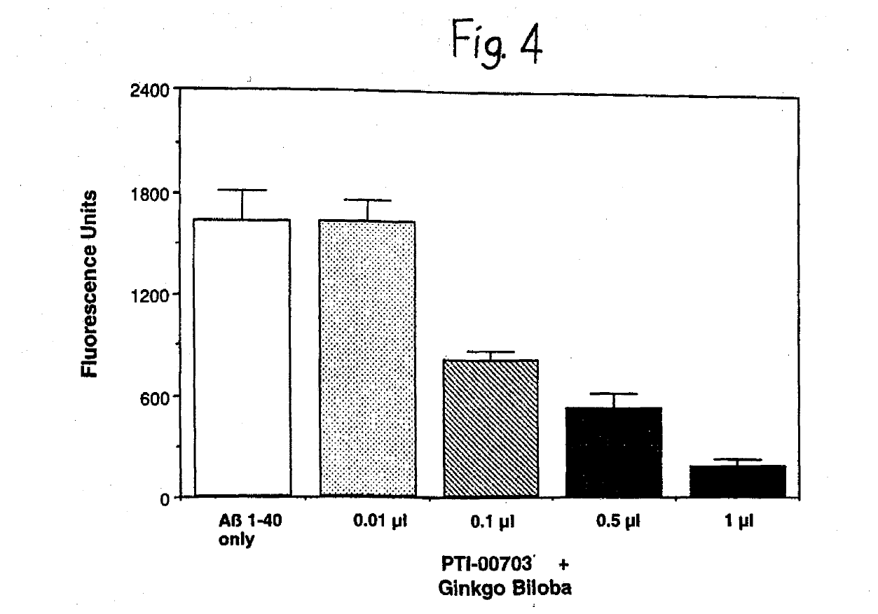

As shown in FIG. 4, water extracts of the test compound caused a dose-dependent dissolution/disruption of pre-formed Aβ 1-40 fibrils within a 2-hour incubation period. For example, 0.5 μl (i.e. 11.5 μg) and 1.0 μl (i.e. 23.0 μg) of water extract caused a significant (p<0.01) 68% and 89% dissolution/disruption of Aβ 1-40 amyloid fibrils, respectively. On the other hand, 0.1 μl (i.e. 2.3 μg) of water extract still caused a significant (p<0.01) 51% dissolution/disruption of Aβ 1-40 amyloid fibrils, whereas 0.01 μl (i.e. 0.23 μg) of water extract did not cause a significant dissolution/disruption of preformed Aβ 1-40 amyloid fibrils. These data demonstrated that the test compound causes a disruption/dissolution of pre-formed Alzheimer's disease Aβ 1-40 amyloid fibrils in a dose-dependent manner. Confirmation of the “dissolution effect” of the test compound, PTI-00703+Gingko biloba, on Alzheimer's disease An 1-40 fibrils was demonstrated by Congo red staining assays, whereby a reduction of congophilia (i.e. red/green birefringence when viewed under polarized light, and which represents a dissolution/disruption of the amyloid fibrillar structure) was observed when Aβ amyloid fibrils were treated for 2 hours (not shown).

### Study 5: Dose-Dependent Disruption of Pre-Formed Alzheimer's Aβ (1-42) Amyloid Fibrils

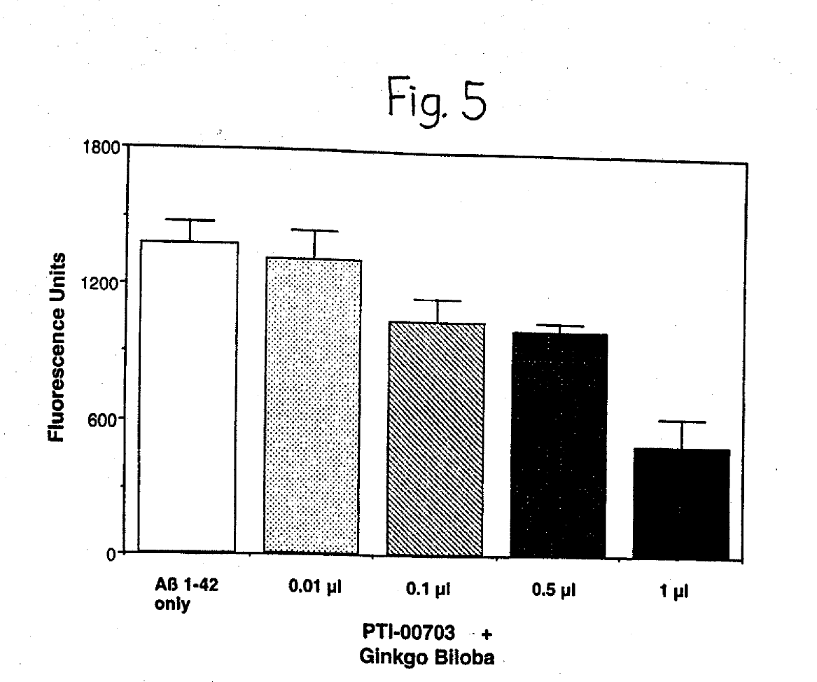

As shown in FIG. 5, the water extracts also caused a dose-dependent dissolution/disruption of pre-formed Aβ 1-42 fibrils within a 2-hour incubation period. For example, 0.5 ul (i.e. 11.5 ug) and 1.0 ul (i.e. 23.0 ug) of water extract caused a significant (p<0.01) 28% and 64% dissolution/disruption of Aβ 1-40 anyloid fibrils, respectively. On the other hand, 0.1 μl (i.e. 2.3 μg) of water extract only caused a 25% dissolution/disruption of Aβ 1-42 amyloid fibrils, whereas 0.01 μl (i.e. 0.23 μg) of water extract did not cause a significant dissolution/disruption of pre-formed Aβ1-42 amyloid fibrils. These data demonstrated that the test compound also causes a disruption/dissolution of pre-formed Alzheimer's disease Aβ 1-42 amyloid fibrils in a dose-dependent manner.

### Comparisons of Data

Comparisons were made to equal volumes (not weights) of water extracts tested; it is important to note that PTI-00703 and PTI-00703 with Gingko biloba were tested at equal volumes of water extracts, and nearly equal weights. Tests results of PTI-00703 alone are not detailed here, having been earlier reported.

### 1) Inhibition of Alzheimer's Amyloid Fibril Formation

This measure of inhibition of amyloid fibril formation is important as an assessment of the compounds potential as a preventative for normal aging and for early stages of Alzheimer's disease.
A) Inhibition at 1 hour (0.5 ul of a water extract): PTI-00703-86% inhibition; PTT-00703 with Gingko biloba—68% inhibition.
B) Inhibition at 1 week (0.5 ul of a water extract): PTI-00703—80% inhibition ; PTI-00703 with Gingko biloba—76% inhibition.

### 2) Inhibition of Alzheimer's Amyloid Fibril Growth

This measure of inhibition of amyloid fibril growth is important as an assessment of the compound's potential as a preventative for normal aging and for early to mid-stages of Alzheimer's disease. PTI-00703—71% inhibition; PTI-00703 with Gingko biloba—82% inhibition.

### 3) Inhibition of Alzheimer's Beta-Amyloid Protein-Glycosaminoglycan Interactions

This measure of inhibition of beta-amyloid protein-glycosaminoglycan interactions is important as an assessment of the compound's potential to inhibit tissue deposition of amyloid, important for normal aging and for all stages of Alzheimer's disease. PTI-00703—97% inhibition; PTI-00703 with Gingko biloba—54% inhibition.

### 4) Dissolution/Disruption of Alzheimer's Disease Aβ 1-40 Fibrils

This measure of dissolution/disruption of pre-formed Aβ 1-40 fibrils is important as an assessment of the compound's potential for later stages of aging and for mid-to-late stages of Alzheimer's disease.
A) Dissolution with 0.5 ul of a water extract: PTI-00703—G3% dissolution; PTI-00703 with Gingko biloba—68% dissolution.
B) Dissolution with 1.0 ul of a water extract: PTI-00703—83% dissolution; PTI-00703 with Gingko biloba—89% dissolution.

### 5) Dissolution/Disruption of Alzheimer's Disease Aβ 1-42 Fibrils

This measure of dissolution/disruption of pre-formed Aβ1-42 fibrils is important as an assessment of the compound's potential use for later stages of aging and for mid-to-late stages of Alzheimer's disease.
A) Dissolution with 0.6 ul of a water extract: PTI-00703-44% dissolution; PTI-00703 with Gingko biloba—28% dissolution.
B) Dissolution with 1.0 ul of a water extract: PTI-00703—82% dissolution; PTI-00703 with Gingko biloba—64% dissolution.

### Synergistic Effects Observed in the Test Formulations

The combination of PTI-00703 and Gingko biloba in the test formulation appears to lead to certain synergistic effects. For example, it appears to be a better inhibitor of amyloid fibril growth (i.e. Aβ-Aβ interactions) than PTI-00703 alone, and a better agent for causing dissolution of pre-formed Alzheimer's disease amyloid fibrils (both Aβ 1-40 and Aβ 1-42) (although statistically these groups may not be different).

Surprisingly, these studies indicate that Gingko biloba alone has no real effects on amyloid fibril growth, or dissolution of pre-formed Aβ 1-40 or Aβ 1-42 amyloid fibrils (not shown). This observation indicates that there are likely true synergistic effects by the combination of PTI-00703 and μl Gingko biloba.

### FURTHER ASPECTS AND UTILIZATIONS OF THE INVENTION

### Therapeutic Applications

One embodiment of the present invention is to formulate prior to administration in a patient, a pharmaceutical blend comprising Uncaria tomentosa in one or more pharmaceutical acceptable carriers, diluents or excipients.

In another preferred embodiment Uncaria tomentosa obtained commercially in any form could be further modulated using suitable carriers, excipients and diluents including lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water syrup, methyl cellulose, methyl and propylhydroxybenzoates, talc, magnesium stearate and mineral oil. The formulations can additionally include lubricating agents, wetting agents, emulsifying and suspending agents, preserving agents, sweetening agents or flavoring agents. The compositions of the invention may be formulated so as to provide quick, sustained or delayed response of the active ingredient after administration to the patient. The compositions are preferably formulated in a unit dosage form, each dosage containing from about 1 to about 10,000 mg of Uncaria tomentosa (or its active ingredients), more usually about 500 to about 2,000 mg of Uncaria tomentosa (or its active ingredients).

However, it will be understood that the therapeutic dosage administered will be determined by the physician in the light of the relevant circumstances including the clinical condition to be treated, the organ or tissues affected or suspected to be affected with amyloid accumulation, and the chosen route of administration. Therefore, the above dosage ranges are not intended to limit the scope of the invention in any way.

The term “unit dosage form” refers to physically discrete units suitable as unitary dosages for human subjects and other mammals, each unit containing a predetermined quantity of active material calculated to produce the desired therapeutic effect, in association with a suitable pharmaceutical carrier.

### INDUSTRIAL APPLICABILITY

Use of extracts from the inner bark and root parts of Uncaria tomentosa, and its blends benefit human patients with Alzheimer's disease and other amyloidoses due to the newly discovered ability of Uncaria tomentosa in combination with the disclosed blended ingredients to inhibit amyloid fibril formation, inhibit amyloid fibril growth, inhibit amyloid-proteoglycan interactions, inhibit amyloid-glycosaminoglyean interactions, and cause dissolution and/or disruption of preformed amyloid fibrils.

### FURTHER EXAMPLES

### Methodology

### Composition Preparation

For these studies, the powder of commercially available standardized extracts within a gelatin capsule of components grape seed extract (GSE, GNC Herbal Plus, Lot#1989EF1980), green tea extract (GTE, Radiance, Ronkonkoma, N.Y.; Lot#65005), gingko biloba extract (GBE, Oregon's Wild Harvest Lot#G001267701), curcumin (CUR, Sigma, St. Louis, Mo.; Lot#104K1088), and PTI-00703 (703), was individually weighed to make 5 mg/ml stock solutions. The solutions were combined to make up 1 ml for each composition (i.e. composition 28: 0.2 ml PTI-703+0.2 ml GTE+0.2 ml GSE+0.2 ml curcumin+0.2 ml dH20=1 ml) or (i.e. composition 7: 0.2 ml PTI-703+0.2 ml GTE+0.6 ml dH20=1 ml), with each individual component contributing 1 mg/ml in concentration to the overall mixture. Distilled water was added to complete any target volume. All of the compositions tested are summarized in Table 1.

TABLE 1
Summary of compositions tested.

| Compo- | |
|---|---|
| sition | |
| No. | Components and Volumes |
| 1 | 0.2 ml 703 + 0.8 ml dH20 |
| 2 | 0.2 ml GSE + 0.8 ml dH20 |
| 3 | 0.2 ml GT + 0.8 ml dH20 |
| 4 | 0.2 ml GB + 0.8 ml dH20 |
| 5 | 0.2 ml CURC + 0.8 ml dH20 |
| 6 | 0.2 ml 703 + 0.2 ml GSE + 0.6 ml dH20 |
| 7 | 0.2 ml 703 + 0.2 ml GT + 0.6 ml dH20 |
| 8 | 0.2 ml 703 + 0.2 ml GB + 0.6 ml dH20 |
| 9 | 0.2 ml 703 + 0.2 ml CUR + 0.6 ml dH20 |
| 10 | 0.2 ml GSE + 0.2 ml GT + 0.6 ml dH20 |
| 11 | 0.2 ml GSE + 0.2 ml GB + 0.6 ml dH20 |
| 12 | 0.2 ml GSE + 0.2 ml CUR + 0.6 ml dH20 |
| 13 | 0.2 ml GT + 0.2 ml GB + 0.6 ml dH20 |
| 14 | 0.2 ml GT + 0.2 ml CUR + 0.6 ml dH20 |
| 15 | 0.2 ml GB + 0.2 ml CUR + 0.6 ml dH20 |
| 16 | 0.2 ml 703 + 0.2 ml GSE + 0.2 ml GT + 0.4 ml dH20 |
| 17 | 0.2 ml 703 + 0.2 ml GSE + 0.2 ml GB + 0.4 ml dH20 |
| 18 | 0.2 ml 703 + 0.2 ml GSE + 0.2 ml CUR + 0.4 ml dH20 |
| 19 | 0.2 ml 703 + 0.2 ml GT + 0.2 ml GB + 0.4 ml dH20 |
| 20 | 0.2 ml 703 + 0.2 ml GT + 0.2 ml CUR + 0.4 ml dH20 |
| 21 | 0.2 ml 703 + 0.2 ml GB + 0.2 ml CUR + 0.4 ml dH20 |
| 22 | 0.2 ml GSE + 0.2 ml GT + 0.2 ml GB + 0.4 ml dH20 |
| 23 | 0.2 ml GSE + 0.2 ml GT + 0.2 ml CUR + 0.4 ml dH20 |
| 24 | 0.2 ml GSE + 0.2 ml GB + 0.2 ml CUR + 0.4 ml dH20 |
| 25 | 0.2 ml GT + 0.2 ml GB + 0.2 ml CUR + 0.4 ml dH20 |
| 26 | 0.2 ml 703 + 0.2 ml GSE + 0.2 ml GB + 0.2 ml CUR + 0.2 ml |
| | dH20 |
| 27 | 0.2 ml 703 + 0.2 ml GSE + 0.2 ml GT + 0.2 ml GB + 0.2 ml |
| | dH20 |
| 28 | 0.2 ml 703 + 0.2 ml GSE + 0.2 ml GT + 0.2 ml CUR + 0.2 ml |
| | dH20 |
| 29 | 0.2 ml 703 + 0.2 ml GT + 0.2 ml GB + 0.2 ml CUR + 0.2 ml |
| | dH20 |
| 30 | 0.2 ml GSE + 0.2 ml GT + 0.2 ml GB + 0.2 ml CUR + 0.2 ml |
| | dH20 |
| 31 | 0.2 ml 703 + 0.2 ml GSE + 0.2 ml GT + 0.2 ml GB + 0.2 ml |
| | CUR |

### Aβ 142 Preparation

1 mg of Aβ 1-42 (Aβ was obtained from rPeptide, Bogart, Ga.; Lot#2180742T) was reconstituted in 1 ml of double distilled water (1 mg/ml solution). 30 μg of Aβ was incubated at 37° C. for 3 days in the absence or presence of one or more blended compositions in 1× Phosphate Buffered Saline (pH 7.4) with 0.02% sodium azide. For all solutions and compositions being tested, there were four plates measured at Aβ: test compound weight/weight ratios of 1:1, 1:0.1, 1:0.01, and 1:0.001. Each of the four different plates with corresponding ratios was analyzed either by Thioflavin T fluorometry or Congo Red Assays.

α-synuclein Preparation

1 mg of α-synuclein (obtained from rPeptide, Bogart, Ga.; Lot#50306AS) was reconstituted in 1.0 ml of 20 mM Sodium Acetate (Sigma lot 67H1002) pH 4.0 (1 mg/ml solution). The α-synuclein was pre-fibrillized by incubating at 37° C. for 4 days at 1400 rpm in an orbital shaker (Labnet Vortemp 56, Woodbridge, N.J.; serial#06040580A). 30 μg of α-Synuclein was then incubated at 37° C. 3 days, in the absence or presence of one or more blended compositions in 1× Phosphate Buffered Saline (pH 7.4) with 0.02% sodium azide. For all solutions and compositions being tested, there were four plates measured at Aβ: test compound weight/weight ratios of 1:1, 1:0.1, 1:0.01, and 1:0.001. Each of the four different plates with corresponding ratios was analyzed either by Thioflavin T fluorometry or Congo Red Assays.

Thioflavin T fluorometry

A previously described method of measuring amyloid fibril formation utilizing Thioflavin T fluorometry (H Naiki et al, Lab. Invest. 65:104-110, 1991; H Levine III, Protein Sci. 2:404-410, 1993; H Levine III, Amyloid: Int. J. Exp. Clin. Invest. 2:1-6, 1995; H Naiki and K. Nakakuki, Lab. Invest. 74:374-383, 1996) was employed to assess whether the above described mixed compositions were capable of causing a disassembly/disruption of Aβ 1-42 amyloid fibrils. Thioflavin T is known to bind to fibrillar amyloid proteins, and an increase in fluorescence correlates with an increase in amyloid fibril formation, whereas a decrease in fluorescence correlates with a decrease in amyloid fibrils due to disassembly and/or disruption. The Aβ protein (1-42) when placed in solution, such as distilled water, tends to spontaneously form amyloid fibrils. Using this sensitive assay, any decreases or increases in fluorescence was previously shown to correlate with a decrease or increase in the amount of amyloid fibrils (H Naiki et al, Lab. Invest. 65:104-110, 1991; H Levine III, Protein Sci. 2:404-410, 1993; H Levine III, Amyloid: Int. I. Exp. Clin. Invest. 2:1-6, 1995; H Naiki and K. Nakakuki, Lab. Invest. 74:374-383, 1996), allowing one to identify and quantitate the extent of potential inhibitors and/or enhancers of Aβ 1-42 amyloid fibrils.

Following 3 days of co-incubation, 50 μl of each incubation mixture was transferred into a 96-well microtiter plate (Whatman Cat# 7701-7350) with 150 μl of distilled water and 50 μl of Thioflavin T solution (500 mM Thioflavin T in 250 mM phosphate buffer, pH 6.8). Using an ELISA plate fluorometer, the fluorescence was read at 485 nm (444 nm excitation wavelength) after subtraction with buffer alone or composition alone, as blank.

### Congo Red Assays

The effectiveness of standardized compositions on disaggregation of Alzheimer's Aβ 1-42 amyloid fibrils was measured by using a Congo red-Aβ spectrophotometric assay (Klunk et al, Anal. Biochem. 266:66-76, 1999).

After three days of incubation of Aβ 1-42 fibrils in the presence or absence of compositions of grape seed extract (GSE), green tea extract (GTE), gingko biloba extract (GBE), curcumin (CUR), and PTI-00703 (703), 200 μl of the incubation mixtures was transferred into Congo red assay plates (Millipore 0.2 μm filter plate; Cat# MSGVN2250). The plates were then vacuum filtered. After the plates were left to dry, 50 μl of Congo red solution (125 uM Congo red in 100 mM Tris+50 mM NaCl pH 7.0) was added to the wells and incubated at room temperature for 10 minutes. Then the plates were washed with 100 μl of 3% MEOH three times, vacuumed between washes and left to dry. The dry plates were scanned into Adobe Photoshop, and the images were imported to Image Quant 5.2 (Molecular Dynamics). Image Quant 5.2 was used to count the number of pixels per area per well on the plates. After washing, any lowering of the Congo red color on the filter in the presence of the test composition (compared to the Congo red staining on the amyloid protein in the absence of the test compound) was indicative of the test compositions ability to diminish/alter the amount of aggregated and congophilic Aβ.

Study 6—Disassembly/Disruption of Aβ 1-42 Amyloid Fibrils Measured using Thioflavin T Assay

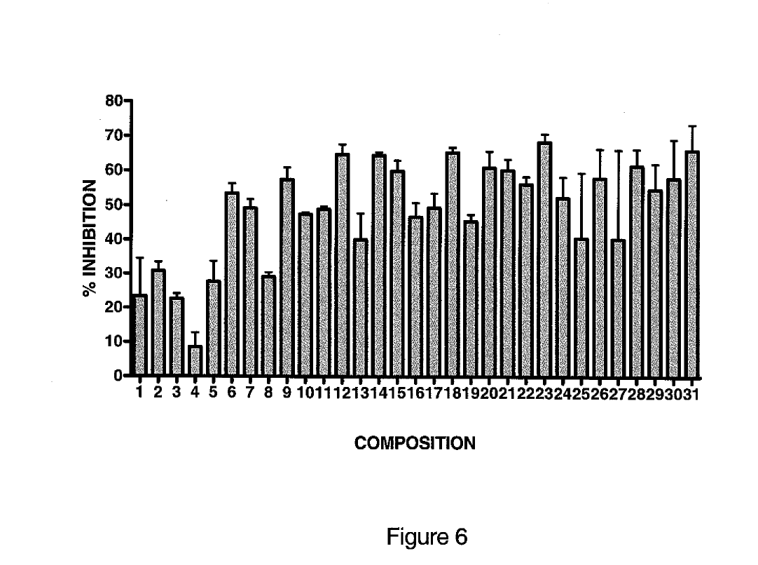

In this study, the compositions and Aβ were prepared as outlined above. Thioflavin T fluorometry was used to measure the disassembly/disruption of Aβ 1-42 amyloid fibrils. The results of this study are summarized in Table 2 and the Aβ: composition ratio of 1:0.1 is shown graphically in FIG. 6.

Compositions 1-5 (one component compositions) displayed variable inhibition ranging from 9 to 31%. No correlation was observed regarding increased efficacy of the composition in its ability to cause disassembly/disruption of Aβ 1-42 amyloid fibrils with increasing number of components in the composition. Unexpectedly, some of the dual component compositions (compositions 9 and 11-15) were just as efficacious as the multi-component compositions. Also unexpected, was the result that dual compositions 9 and 11-15 displayed better than expected results when compared to a projected total based on addition of single component inhibition rates (shown in table 3 for 1:0.1 ratio).

TABLE 2
% inhibition of disassembly/disruption of Aβ 1-42
amyloid fibrils measured using Thio T assay.

| Composition and | | | | % Std. |
|---|---|---|---|---|
| Ratio | Ave | Std. dev | % Inhibition | dev |
| Aβ 1-42 only | 2961.5 | 76.5 | 0.0 | 5.02 |
| 1 1:1 | 718.0 | 26.6 | 75.8 | 0.90 |
| 1 1:0.1 | 2267.7 | 327.8 | 23.4 | 11.07 |
| 1 1:0.01 | 2500.6 | 126.8 | 15.6 | 4.28 |
| 2 1:1 | 436.9 | 77.8 | 85.2 | 2.63 |
| 2 1:0.1 | 2048.7 | 77.1 | 30.8 | 2.60 |
| 2 1:0.01 | 2356.8 | 147.4 | 20.4 | 4.98 |
| 3 1:1 | 564.9 | 48.1 | 80.9 | 1.62 |
| 3 1:0.1 | 2293.5 | 45.0 | 22.6 | 1.52 |
| 3 1:0.01 | 2672.9 | 129.9 | 9.7 | 4.38 |
| 4 1:1 | 1157.5 | 57.7 | 60.9 | 1.95 |
| 4 1:0.1 | 2710.3 | 124.1 | 8.5 | 4.19 |
| 4 1:0.01 | 2765.3 | 113.1 | 6.6 | 3.82 |
| 5 1:1 | 746.6 | 26.6 | 74.8 | 0.90 |
| 5 1:0.1 | 2141.6 | 176.5 | 27.7 | 5.96 |
| 5 1:0.01 | 2382.1 | 69.9 | 19.6 | 2.36 |
| 6 1:1 | 242.8 | 33.4 | 91.8 | 1.13 |
| 6 1:0.1 | 1377.0 | 83.6 | 53.5 | 2.82 |
| 6 1.0.01 | 2277.3 | 95.6 | 23.1 | 3.23 |
| 7 1:1 | 255.5 | 7.7 | 91.4 | 0.26 |
| 7 1:0.1 | 1503.4 | 77.6 | 49.2 | 2.62 |
| 7 1:0.01 | 2190.1 | 51.2 | 26.0 | 1.73 |
| 8 1:1 | 457.4 | 39.3 | 84.6 | 1.33 |
| 8 1:0.1 | 2100.5 | 38.3 | 29.1 | 1.29 |
| 8 1:0.01 | 2760.3 | 277.5 | 6.8 | 9.37 |
| 9 1:1 | 424.1 | 4.4 | 85.7 | 0.15 |
| 9 1:0.1 | 1258.5 | 104.6 | 57.5 | 3.53 |
| 9 1:0.01 | 2287.6 | 307.4 | 22.8 | 10.38 |
| 10 1:1 | 150.8 | 16.0 | 94.9 | 0.54 |
| 10 1:0.1 | 1554.7 | 15.5 | 47.5 | 0.52 |
| 10 1:0.01 | 2501.0 | 187.6 | 15.5 | 6.33 |
| 11 1:1 | 265.8 | 19.3 | 91.0 | 0.65 |
| 11 1:0.1 | 1510.7 | 23.5 | 49.0 | 0.79 |
| 11 1:0.01 | 2364.6 | 86.3 | 20.2 | 2.91 |
| 12 1:1 | 304.1 | 44.9 | 89.7 | 1.52 |
| 12 1:0.1 | 1039.8 | 84.6 | 64.9 | 2.86 |
| 12 1:0.01 | 2228.1 | 11.7 | 24.8 | 0.39 |
| 13 1:1 | 347.5 | 34.6 | 88.3 | 1.17 |
| 13 1:0.1 | 1770.9 | 226.0 | 40.2 | 7.63 |
| 13 1:0.01 | 2441.6 | 101.6 | 17.6 | 3.43 |
| 14 1:1 | 304.1 | 17.7 | 89.7 | 0.60 |
| 14 1:0.1 | 1047.9 | 26.6 | 64.6 | 0.90 |
| 14 1:0.01 | 1974.2 | 127.2 | 33.3 | 4.30 |
| 15 1:1 | 618.3 | 59.5 | 79.1 | 2.01 |
| 15 1:0.1 | 1181.5 | 91.8 | 60.1 | 3.10 |
| 15 1:0.01 | 2253.9 | 185.2 | 23.9 | 6.25 |
| 16 1:1 | 170.3 | 13.4 | 94.3 | 0.45 |
| 16 1:0.1 | 1576.7 | 122.6 | 46.8 | 4.14 |
| 16 1:0.01 | 2509.5 | 126.9 | 15.3 | 4.29 |
| 17 1:1 | 239.9 | 13.4 | 91.9 | 0.45 |
| 17 1:0.1 | 1496.9 | 123.6 | 49.5 | 4.17 |
| 17 1:0.01 | 2520.0 | 41.2 | 14.9 | 1.39 |
| 18 1:1 | 234.7 | 34.9 | 92.1 | 1.18 |
| 18 1:0.1 | 1021.0 | 42.6 | 65.5 | 1.44 |
| 18 1:0.01 | 2225.3 | 355.9 | 24.9 | 12.02 |
| 19 1:1 | 247.6 | 23.2 | 91.6 | 0.78 |
| 19 1:0.1 | 1612.5 | 58.3 | 45.6 | 1.97 |
| 19 1:0.01 | 2677.2 | 228.5 | 9.6 | 7.72 |
| 20 1:1 | 276.1 | 24.9 | 90.7 | 0.84 |
| 20 1:0.1 | 1152.1 | 141.9 | 61.1 | 4.79 |
| 20 1:0.01 | 2401.1 | 151.0 | 18.9 | 5.10 |
| 21 1:1 | 402.4 | 15.5 | 86.4 | 0.52 |
| 21 1:0.1 | 1172.5 | 93.9 | 60.4 | 3.17 |
| 21 1:0.01 | 2362.4 | 219.2 | 20.2 | 7.40 |
| 22 1:1 | 208.9 | 16.1 | 92.9 | 0.55 |
| 22 1:0.1 | 1291.0 | 64.0 | 56.4 | 2.16 |
| 22 1:0.01 | 2235.6 | 85.5 | 24.5 | 2.89 |
| 23 1:1 | 206.4 | 17.9 | 93.0 | 0.60 |
| 23 1:0.1 | 928.6 | 69.9 | 68.6 | 2.36 |
| 23 1:0.01 | 2093.2 | 54.5 | 29.3 | 1.84 |
| 24 1:1 | 276.0 | 15.5 | 90.7 | 0.52 |
| 24 1:0.1 | 1406.7 | 178.4 | 52.5 | 6.02 |
| 24 1:0.01 | 2457.5 | 81.8 | 17.0 | 2.76 |
| 25 1:1 | 358.6 | 11.8 | 87.9 | 0.40 |
| 25 1:0.1 | 1753.9 | 561.1 | 40.8 | 18.95 |
| 25 1:0.01 | 2375.1 | 169.5 | 19.8 | 5.72 |
| 26 1:1 | 211.6 | 31.3 | 92.9 | 1.06 |
| 26 1:0.1 | 1236.9 | 251.0 | 58.2 | 8.47 |
| 26 1:0.01 | 2297.5 | 144.1 | 22.4 | 4.87 |
| 27 1:1 | 134.2 | 4.5 | 95.5 | 0.15 |
| 27 1:0.1 | 1761.5 | 768.5 | 40.5 | 25.95 |
| 27 1:0.01 | 2315.7 | 23.9 | 21.8 | 0.81 |
| 28 1:1 | 175.4 | 23.3 | 94.1 | 0.79 |
| 28 1:0.1 | 1131.4 | 143.0 | 61.8 | 4.83 |
| 28 1:0.01 | 2328.7 | 178.6 | 21.4 | 6.03 |
| 29 1:1 | 201.2 | 7.8 | 93.2 | 0.26 |
| 29 1:0.1 | 1332.2 | 218.9 | 55.0 | 7.39 |
| 29 1:0.01 | 2300.6 | 135.1 | 22.3 | 4.56 |
| 30 1:1 | 185.7 | 4.4 | 93.7 | 0.15 |
| 30 1:0.1 | 1239.4 | 336.0 | 58.2 | 11.35 |
| 30 1:0.01 | 2023.5 | 194.2 | 31.7 | 6.56 |
| 31 1:1 | 139.3 | 11.8 | 95.3 | 0.40 |
| 31 1:0.1 | 995.3 | 222.6 | 66.4 | 7.52 |
| 31 1:0.01 | 1840.3 | 296.1 | 37.9 | 10.00 |

TABLE 3
Comparison of expected and observed % inhibition
of disassembly/disruption of Aβ 1-42 amyloid fibrils
measured using Thio T assay at 1:0.1.

| No. | Composition | Expected | Observed |
|---|---|---|---|
| 1 | 703 | — | 23 |
| 2 | GSE | — | 31 |
| 3 | GTE | — | 23 |
| 4 | GBE | — | 9 |
| 5 | CUR | — | 28 |
| 6 | 703 + GSE | 54 | 54 |
| 7 | 703 + GTE | 46 | 49 |
| 8 | 703 + GBE | 32 | 29 |
| 9 | 703 + CUR | 51 | 58 |
| 10 | GSE + GTE | 54 | 48 |
| 11 | GSE + GBE | 40 | 49 |
| 12 | GSE + CUR | 59 | 65 |
| 13 | GTE + GBE | 32 | 40 |
| 14 | GTE + CUR | 51 | 65 |
| 15 | GBE + CUR | 37 | 60 |
| 16 | 703 + GSE + GTE | 77 | 47 |
| 17 | 703 + GSE + GBE | 63 | 50 |
| 18 | 703 + GSE + CUR | 72 | 66 |
| 19 | 703 + GTE + GBE | 55 | 46 |
| 20 | 703 + GTE + CUR | 74 | 61 |
| 21 | 703 + GBE + CUR | 60 | 60 |
| 22 | GSE + GTE + GBE | 63 | 56 |
| 23 | GSE + GTE + CUR | 82 | 69 |
| 24 | GSE + GBE + CUR | 68 | 53 |
| 25 | GTE + GBE + CUR | 60 | 41 |
| 26 | 703 + GSE + GBE + CUR | 91 | 58 |
| 27 | 703 + GSE + GTE + GBE | 86 | 41 |
| 28 | 703 + GSE + GTE + CUR | 105 | 62 |
| 29 | 703 + GTE + GBE + CUR | 83 | 55 |
| 30 | GSE + GTE + GBE + CUR | 91 | 58 |
| 31 | 703 + GSE + GTE + GBE + CUR | 114 | 66 |

One might expect that compositions comprising more than one component would show at least additive effects in terms of overall efficacy of the composition in its ability to cause disassembly/disruption of Aβ 1-42 amyloid fibrils. However, this was not generally the case. In FIG. 6 and table 3 for example, only two compositions had equivalent observed and expected results (compositions 6 and 21). In seventeen of the thirty one compositions (compositions 7, 10, 16-20 and 22-31), the expected results far exceeded the observed results and it was therefore surprising that in only six cases (compositions 9, and 11-15) the actual observed results exceeded the expected results

### Study 7—Disaggregation of Aβ 1-42 Amyloid Fibrils Measured by Congo Red Assay

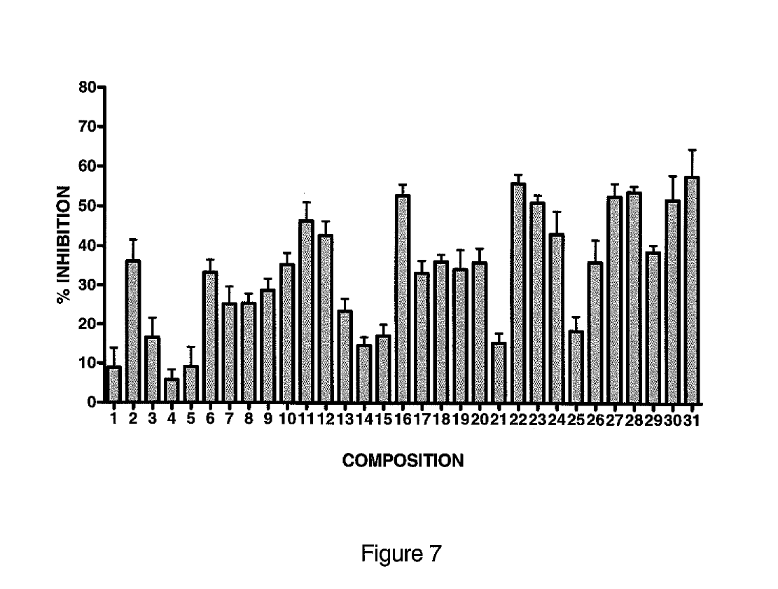

In this study, the compositions and Aβ were prepared as outlined above. The Congo red-Aβ spectrophotometric assay was used to measure the disaggregation of Aβ 1-42 amyloid fibrils. The results of this study are summarized in Table 4 and the Aβ: composition ratio of 1:1 shown graphically in FIG. 7. Compositions 1-5 (one component compositions) displayed more variable inhibition ranges than those detected in the Thio T assay. Again, no correlation was observed regarding increased efficacies in the disassembly/disruption of Aβ 1-42 amyloid fibrils with increasing number of components in the composition. Unexpectedly, some of the dual and tri-component compositions (compositions 9, 11, 13, 15, 19 and 20) were just as efficacious as the multi-component compositions. Also unexpected, was the result that dual compositions 9, 11, 13, 15, 19 and 20 displayed better than expected results when compared to a total based on addition of single component inhibition rates (shown in table 5 for 1:1 ratio).

TABLE 4
% inhibition of disaggregation of Aβ 1-42 amyloid
fibrils measured using Congo Red assay

| Composition and | | | | |
|---|---|---|---|---|
| Ratio | Average | Std. dev | % Inhibition | % Std. dev |
| A□42 only | 210678 | 4294 | 0.0 | 2.04 |
| 1 1:1 | 191895 | 10519 | 8.9 | 4.99 |
| 1 1:0.1 | 216695 | 6339 | −2.9 | 3.01 |
| 1 1:0.01 | 244132 | 2302 | −15.9 | 1.09 |
| 2 1:1 | 134642 | 11410 | 36.1 | 5.42 |
| 2 1:0.1 | 190544 | 3192 | 9.6 | 1.52 |
| 2 1:0.01 | 220066 | 13465 | −4.5 | 6.39 |
| 3 1:1 | 175705 | 10555 | 16.6 | 5.01 |
| 3 1:0.1 | 222774 | 1707 | −5.7 | 0.81 |
| 3 1:0.01 | 238179 | 5500 | −13.1 | 2.61 |
| 4 1:1 | 198410 | 5317 | 5.8 | 2.52 |
| 4 1:0.1 | 209858 | 8654 | 0.4 | 4.11 |
| 4 1:0.01 | 226811 | 2010 | −7.7 | 0.95 |
| 5 1:1 | 191414 | 10579 | 9.1 | 5.02 |
| 5 1:0.1 | 204491 | 4023 | 2.9 | 1.91 |
| 5 1:0.01 | 207666 | 1454 | 1.4 | 0.69 |
| 6 1:1 | 140596 | 6800 | 33.3 | 3.23 |
| 6 1:0.1 | 174694 | 7670 | 17.1 | 3.64 |
| 6 1:0.01 | 241403 | 5333 | −14.6 | 2.53 |
| 7 1:1 | 157659 | 9391 | 25.2 | 4.46 |
| 7 1:0.1 | 204813 | 2201 | 2.8 | 1.04 |
| 7 1:0.01 | 210949 | 2103 | −0.1 | 1.00 |
| 8 1:1 | 157189 | 5167 | 25.4 | 2.45 |
| 8 1:0.1 | 180133 | 5587 | 14.5 | 2.65 |
| 8 1:0.01 | 202245 | 4827 | 4.0 | 2.29 |
| 9 1:1 | 150213 | 6270 | 28.7 | 2.98 |
| 9 1:0.1 | 169443 | 219 | 19.6 | 0.10 |
| 9 1:0.01 | 204694 | 3873 | 2.8 | 1.84 |
| 10 1:1 | 136117 | 6117 | 35.4 | 2.90 |
| 10 1:0.1 | 178174 | 5294 | 15.4 | 2.51 |
| 10 1:0.01 | 223589 | 3138 | −6.1 | 1.49 |
| 11 1:1 | 112981 | 9947 | 46.4 | 4.72 |
| 11 1:0.1 | 168169 | 9668 | 20.2 | 4.59 |
| 11 1:0.01 | 212906 | 4605 | −1.1 | 2.19 |
| 12 1:1 | 120463 | 7563 | 42.8 | 3.59 |
| 12 1:0.1 | 161709 | 5607 | 23.2 | 2.66 |
| 12 1:0.01 | 212541 | 17750 | −0.9 | 8.43 |
| 13 1:1 | 161198 | 6699 | 23.5 | 3.18 |
| 13 1:0.1 | 211408 | 8462 | −0.3 | 4.02 |
| 13 1:0.01 | 226050 | 11511 | −7.3 | 5.46 |
| 14 1:1 | 179634 | 4504 | 14.7 | 2.14 |
| 14 1:0.1 | 175324 | 11033 | 16.8 | 5.24 |
| 14 1:0.01 | 213308 | 6377 | −1.2 | 3.03 |
| 15 1:1 | 174417 | 6099 | 17.2 | 2.89 |
| 15 1:0.1 | 180199 | 13245 | 14.5 | 6.29 |
| 15 1:0.01 | 208898 | 12362 | 0.8 | 5.87 |
| 16 1:1 | 99207 | 5788 | 52.9 | 2.75 |
| 16 1:0.1 | 187502 | 8862 | 11.0 | 4.21 |
| 16 1:0.01 | 222349 | 10007 | −5.5 | 4.75 |
| 17 1:1 | 140467 | 6773 | 33.3 | 3.22 |
| 17 1:0.1 | 178672 | 9988 | 15.2 | 4.74 |
| 17 1:0.01 | 206907 | 4942 | 1.8 | 2.35 |
| 18 1:1 | 134155 | 3641 | 36.3 | 1.73 |
| 18 1:0.1 | 163658 | 3231 | 22.3 | 1.53 |
| 18 1:0.01 | 212163 | 4328 | −0.7 | 2.05 |
| 19 1:1 | 138382 | 10428 | 34.3 | 4.95 |
| 19 1:0.1 | 210070 | 5599 | 0.3 | 2.66 |
| 19 1:0.01 | 223313 | 4749 | −6.0 | 2.25 |
| 20 1:1 | 134749 | 7595 | 36.0 | 3.61 |
| 20 1:0.1 | 194651 | 3984 | 7.6 | 1.89 |
| 20 1:0.01 | 221198 | 16254 | −5.0 | 7.72 |
| 21 1:1 | 178185 | 5151 | 15.4 | 2.44 |
| 21 1:0.1 | 181774 | 21667 | 13.7 | 10.28 |
| 21 1:0.01 | 228148 | 8975 | −8.3 | 4.26 |
| 21 1:1 | 92727 | 5003 | 56.0 | 2.37 |
| 22 1:0.1 | 197377 | 4581 | 6.3 | 2.17 |
| 22 1:0.01 | 206086 | 7595 | 2.2 | 3.60 |
| 23 1:1 | 102848 | 4067 | 51.2 | 1.93 |
| 23 1:0.1 | 179558 | 2180 | 14.8 | 1.03 |
| 23 1:0.01 | 204853 | 6499 | 2.8 | 3.08 |
| 24 1:1 | 119379 | 12125 | 43.3 | 5.76 |
| 24 1:0.1 | 142166 | 17077 | 32.5 | 8.11 |
| 24 1:0.01 | 216726 | 2730 | −2.9 | 1.30 |
| 25 1:1 | 171780 | 7843 | 18.5 | 3.72 |
| 25 1:0.1 | 200293 | 21084 | 4.9 | 10.01 |
| 25 1:0.01 | 208453 | 9443 | 1.1 | 4.48 |
| 26 1:1 | 134347 | 11929 | 36.2 | 5.66 |
| 26 1:0.1 | 145584 | 7077 | 30.9 | 3.36 |
| 26 1:0.01 | 206162 | 10722 | 2.1 | 5.09 |
| 27 1:1 | 99364 | 7055 | 52.8 | 3.35 |
| 27 1:0.1 | 178415 | 19942 | 15.3 | 9.47 |
| 27 1:0.01 | 204932 | 6597 | 2.7 | 3.13 |
| 28 1:1 | 96869 | 3137 | 54.0 | 1.49 |
| 28 1:0.1 | 151696 | 19374 | 28.0 | 9.20 |
| 28 1:0.01 | 241751 | 2785 | −14.7 | 1.32 |
| 29 1:1 | 128679 | 3621 | 38.9 | 1.72 |
| 29 1:0.1 | 187437 | 29608 | 11.0 | 14.05 |
| 29 1:0.01 | 229180 | 11283 | −8.8 | 5.36 |
| 30 1:1 | 101228 | 13239 | 52.0 | 6.28 |
| 30 1:0.1 | 156145 | 27342 | 25.9 | 12.98 |
| 30 1:0.01 | 200830 | 12451 | 4.7 | 5.91 |
| 31 1:1 | 88556 | 14703 | 58.0 | 6.98 |
| 31 1:0.1 | 151300 | 18011 | 28.2 | 8.55 |
| 31 1:0.01 | 210123 | 16360 | 0.3 | 7.77 |

TABLE 5
Comparison of expected and observed % inhibition
of disaggregation of Aβ 1-42 amyloid fibrils measured
using Congo Red assay at 1:1.

| No. | Composition | Expected | Observed |
|---|---|---|---|
| 1 | 703 | | |
| 2 | GSE | | |
| 3 | GTE | | |
| 4 | GBE | | |
| 5 | CUR | | |
| 6 | 703 + GSE | 45 | 33 |
| 7 | 703 + GTE | 26 | 25 |
| 8 | 703 + GBE | 15 | 25 |
| 9 | 703 + CUR | 18 | 29 |
| 10 | GSE + GTE | 53 | 35 |
| 11 | GSE + GBE | 42 | 46 |
| 12 | GSE + CUR | 45 | 43 |
| 13 | GTE + GBE | 23 | 24 |
| 14 | GTE + CUR | 26 | 15 |
| 15 | GBE + CUR | 15 | 17 |
| 16 | 703 + GSE + GTE | 62 | 53 |
| 17 | 703 + GSE + GBE | 51 | 33 |
| 18 | 703 + GSE + CUR | 54 | 36 |
| 19 | 703 + GTE + GBE | 32 | 34 |
| 20 | 703 + GTE + CUR | 35 | 36 |
| 21 | 703 + GBE + CUR | 24 | 15 |
| 22 | GSE + GTE + GBE | 59 | 56 |
| 23 | GSE + GTE + CUR | 62 | 51 |
| 24 | GSE + GBE + CUR | 51 | 43 |
| 25 | GTE + GBE + CUR | 32 | 19 |
| 26 | 703 + GSE + GBE + CUR | 60 | 36 |
| 27 | 703 + GSE + GTE + GBE | 68 | 53 |
| 28 | 703 + GSE + GTE + CUR | 71 | 54 |
| 29 | 703 + GTE + GBE + CUR | 41 | 39 |
| 30 | GSE + GTE + GBE + CUR | 68 | 52 |
| 31 | 703 + GSE + GTE + GBE + | 77 | 58 |
| | CUR | | |

Again one might expect that compositions comprising more than one component would show at least additive effects in terms of overall efficacy of the composition in its ability to cause disaggregation of Aβ 1-42 amyloid fibrils. However, this was not generally the case. For the results measured by Congo Red and shown in FIG. 7 and table 5, for example, none of the compositions had equivalent observed and expected results. In nineteen of the thirty one compositions (compositions 6, 7, 10, 12, 14, 16-18 and 21-31), the expected results far exceeded the observed results and it was therefore surprising that in only seven cases (compositions 8, 9, 11, 13, 15, 19 and 20) the actual observed results exceeded the expected results.

Study 8—Disassembly/Disruption of α-Synuclein Amyloid Fibrils Measured using Thioflavin T Assay

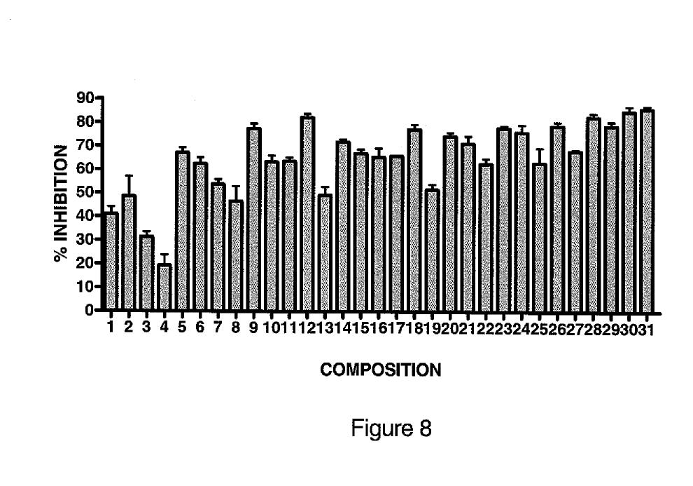

In this study, the compositions and α-synuclein were prepared as outlined above. Thioflavin T fluorometry was used to measure the disassembly/disruption of α-synuclein amyloid fibrils. The results of this study are summarized in Table 6 and the α-synuclein: composition ratio of 1:0.1 is shown graphically in FIG. 8.

Compositions 1-5 (one component compositions) displayed variable inhibition rates ranging from 19-67%. Again, no correlation was observed regarding increased efficacies in the disassembly/disruption of α-synuclein amyloid fibrils with increasing number of components in the composition. Unexpectedly, all of the observed efficacy rates were considerably less than expected when compared to a total based on addition of single component inhibition rates (shown in table 7 for 1:0.1 ratio).

TABLE 6
% inhibition of disassembly/disruption of α-synuclein
amyloid fibrils measured using Thio T assay

| | | | | % Std. |
|---|---|---|---|---|
| Composition &Ratio | Ave | Std. dev | % Inhibition | dev |
| a-Syn only | 736.8 | 14.2 | 0.0 | 3.85 |
| 1 1:1 | 201.7 | 21.3 | 72.6 | 2.90 |
| 1 1:0.1 | 435.1 | 23.3 | 40.9 | 3.16 |
| 1 1:0.01 | 576.7 | 67.6 | 21.7 | 9.18 |
| 2 1:1 | 150.6 | 23.3 | 79.6 | 3.17 |
| 2 1:0.1 | 379.1 | 62.2 | 48.5 | 8.44 |
| 2 1:0.01 | 627.1 | 26.8 | 14.9 | 3.63 |
| 3 1:1 | 196.3 | 16.8 | 73.4 | 2.28 |
| 3 1:0.1 | 506.4 | 15.9 | 31.3 | 2.15 |
| 3 1:0.01 | 621.9 | 70.1 | 15.6 | 9.51 |
| 4 1:1 | 330.8 | 20.3 | 55.1 | 2.75 |
| 4 1:0.1 | 595.4 | 33.3 | 19.2 | 4.52 |
| 4 1:0.01 | 715.3 | 30.8 | 2.9 | 4.18 |
| 5 1:1 | 180.2 | 18.6 | 75.5 | 2.52 |
| 5 1:0.1 | 241.7 | 15.9 | 67.2 | 2.16 |
| 5 1:0.01 | 289.6 | 33.0 | 60.7 | 4.47 |
| 6 1:1 | 126.4 | 29.1 | 82.8 | 3.95 |
| 6 1:0.1 | 277.3 | 20.2 | 62.4 | 2.74 |
| 6 1:0.01 | 513.7 | 30.6 | 30.3 | 4.15 |
| 7 1:1 | 142.6 | 8.1 | 80.6 | 1.09 |
| 7 1:0.1 | 341.0 | 15.9 | 53.7 | 2.16 |
| 7 1:0.01 | 566.6 | 19.0 | 23.1 | 2.58 |
| 8 1:1 | 158.7 | 12.3 | 78.5 | 1.67 |
| 8 1:0.1 | 394.4 | 46.6 | 46.5 | 6.33 |
| 8 1:0.01 | 634.8 | 34.1 | 13.8 | 4.63 |
| 9 1:1 | 69.9 | 20.3 | 90.5 | 2.76 |
| 9 1:0.1 | 165.4 | 15.9 | 77.6 | 2.15 |
| 9 1:0.01 | 380.3 | 61.8 | 48.4 | 8.39 |
| 10 1:1 | 69.9 | 9.4 | 90.5 | 1.27 |
| 10 1:0.1 | 269.7 | 20.2 | 63.4 | 2.74 |
| 10 1:0.01 | 558.9 | 28.4 | 24.1 | 3.85 |
| 11 1:1 | 102.2 | 28.3 | 86.1 | 3.85 |
| 11 1:0.1 | 267.2 | 11.7 | 63.7 | 1.58 |
| 11 1:0.01 | 561.6 | 8.7 | 23.8 | 1.18 |
| 12 1:1 | 86.1 | 12.4 | 88.3 | 1.68 |
| 12 1:0.1 | 129.8 | 11.7 | 82.4 | 1.58 |
| 12 1:0.01 | 402.9 | 19.0 | 45.3 | 2.58 |
| 13 1:1 | 113.0 | 4.7 | 84.7 | 0.63 |
| 13 1:0.1 | 374.1 | 26.8 | 49.2 | 3.64 |
| 13 1:0.01 | 599.2 | 85.5 | 18.7 | 11.61 |
| 14 1:1 | 67.3 | 9.3 | 90.9 | 1.26 |
| 14 1:0.1 | 206.1 | 8.8 | 72.0 | 1.20 |
| 14 1:0.01 | 334.9 | 19.0 | 54.5 | 2.58 |
| 15 1:1 | 110.3 | 12.3 | 85.0 | 1.67 |
| 15 1:0.1 | 241.7 | 11.7 | 67.2 | 1.58 |
| 15 1:0.01 | 360.1 | 17.4 | 51.1 | 2.37 |
| 16 1:1 | 94.3 | 11.7 | 87.2 | 1.58 |
| 16 1:0.1 | 253.3 | 27.7 | 65.6 | 3.76 |
| 16 1:0.01 | 574.0 | 33.2 | 22.1 | 4.50 |
| 17 1:1 | 104.5 | 30.6 | 85.8 | 4.15 |
| 17 1:0.1 | 250.8 | 0.0 | 66.0 | 0.00 |
| 17 1:0.01 | 540.9 | 59.4 | 26.6 | 8.07 |
| 18 1:1 | 91.7 | 20.2 | 87.6 | 2.74 |
| 18 1:0.1 | 166.3 | 15.4 | 77.4 | 2.08 |
| 18 1:0.01 | 349.5 | 34.5 | 52.6 | 4.69 |
| 19 1:1 | 119.8 | 26.8 | 83.7 | 3.64 |
| 19 1:0.1 | 355.7 | 16.0 | 51.7 | 2.17 |
| 19 1:0.01 | 612.4 | 24.7 | 16.9 | 3.35 |
| 20 1:1 | 81.6 | 11.7 | 88.9 | 1.58 |
| 20 1:0.1 | 186.8 | 11.7 | 74.6 | 1.59 |
| 20 1:0.01 | 390.4 | 20.2 | 47.0 | 2.74 |
| 21 1:1 | 89.1 | 11.7 | 87.9 | 1.58 |
| 21 1:0.1 | 209.8 | 23.4 | 71.5 | 3.18 |
| 21 1:0.01 | 380.2 | 40.5 | 48.4 | 5.50 |
| 22 1:1 | 104.5 | 8.8 | 85.8 | 1.20 |
| 22 1:0.1 | 273.7 | 16.0 | 62.8 | 2.17 |
| 22 1:0.01 | 543.4 | 0.0 | 26.2 | 0.00 |
| 23 1:1 | 68.8 | 15.9 | 90.7 | 2.16 |
| 23 1:0.1 | 161.2 | 7.7 | 78.1 | 1.05 |
| 23 1:0.01 | 431.2 | 11.7 | 41.5 | 1.58 |
| 24 1:1 | 84.1 | 20.2 | 88.6 | 2.74 |
| 24 1:0.1 | 174.0 | 23.0 | 76.4 | 3.12 |
| 24 1:0.01 | 456.7 | 42.6 | 38.0 | 5.78 |
| 25 1:1 | 114.7 | 15.3 | 84.4 | 2.08 |
| 25 1:0.1 | 271.2 | 47.9 | 63.2 | 6.51 |
| 25 1:0.01 | 410.7 | 137.2 | 44.3 | 18.62 |
| 26 1:1 | 86.6 | 11.7 | 88.2 | 1.58 |
| 26 1:0.1 | 153.5 | 11.7 | 79.2 | 1.59 |
| 26 1:0.01 | 408.2 | 31.0 | 44.6 | 4.20 |
| 27 1:1 | 76.4 | 13.3 | 89.6 | 1.80 |
| 27 1:0.1 | 232.8 | 4.4 | 68.4 | 0.60 |
| 27 1:0.01 | 487.3 | 4.4 | 33.9 | 0.60 |
| 28 1:1 | 61.1 | 15.9 | 91.7 | 2.16 |
| 28 1:0.1 | 125.4 | 11.8 | 83.0 | 1.60 |
| 28 1:0.01 | 308.7 | 7.7 | 58.1 | 1.04 |
| 29 1:1 | −12.7 | 17.7 | 101.7 | 2.40 |
| 29 1:0.1 | 153.5 | 13.3 | 79.2 | 1.80 |
| 29 1:0.01 | 385.3 | 11.7 | 47.7 | 1.58 |
| 30 1:1 | 63.7 | 11.7 | 91.3 | 1.58 |
| 30 1:0.1 | 107.4 | 15.4 | 85.4 | 2.08 |
| 30 1:0.01 | 375.1 | 26.8 | 49.1 | 3.64 |
| 31 1:1 | 51.0 | 28.9 | 93.1 | 3.93 |
| 31 1:0.1 | 99.8 | 8.8 | 86.5 | 1.20 |
| 31 1:0.01 | 347.0 | 35.4 | 52.9 | 4.80 |

TABLE 7
Comparison of expected and observed % inhibition
of disassembly/disruption of α-synuclein fibrils measured
using Thio T assay at 1:0.1.

| No. | Composition | Expected | Observed |
|---|---|---|---|
| 1 | 703 | | 41 |
| 2 | GSE | | 49 |
| 3 | GTE | | 31 |
| 4 | GBE | | 19 |
| 5 | CUR | | 67 |
| 6 | 703 + GSE | 90 | 62 |
| 7 | 703 + GTE | 72 | 54 |
| 8 | 703 + GBE | 60 | 47 |
| 9 | 703 + CUR | 108 | 78 |
| 10 | GSE + GTE | 80 | 63 |
| 11 | GSE + GBE | 68 | 64 |
| 12 | GSE + CUR | 116 | 82 |
| 13 | GTE + GBE | 50 | 49 |
| 14 | GTE + CUR | 98 | 72 |
| 15 | GBE + CUR | 86 | 67 |
| 16 | 703 + GSE + GTE | 121 | 66 |
| 17 | 703 + GSE + GBE | 109 | 66 |
| 18 | 703 + GSE + CUR | 157 | 77 |
| 19 | 703 + GTE + GBE | 91 | 52 |
| 20 | 703 + GTE + CUR | 139 | 75 |
| 21 | 703 + GBE + CUR | 127 | 72 |
| 22 | GSE + GTE + GBE | 99 | 63 |
| 23 | GSE + GTE + CUR | 147 | 78 |
| 24 | GSE + GBE + CUR | 135 | 76 |
| 25 | GTE + GBE + CUR | 117 | 63 |
| 26 | 703 + GSE + GBE + CUR | 176 | 79 |
| 27 | 703 + GSE + GTE + GBE | 140 | 68 |
| 28 | 703 + GSE + GTE + CUR | 188 | 83 |
| 29 | 703 + GTE + GBE + CUR | 158 | 79 |
| 30 | GSE + GTE + GBE + CUR | 166 | 85 |
| 31 | 703 + GSE + GTE + GBE + | 207 | 87 |
| | CUR | | |

One might expect that compositions comprising more than one component would show at least additive effects in terms of overall efficacy of the composition in its ability to cause disassembly/disruption of α-synuclein fibrils. However, this was not generally the case. As shown in FIG. 8 and table 7, for example, none of the compositions had equivalent observed and expected results. In all cases, the expected results far exceeded the observed results.

### Study 9—Disaggregation of α-Synuclein Fibrils Measured by Congo Red Assay

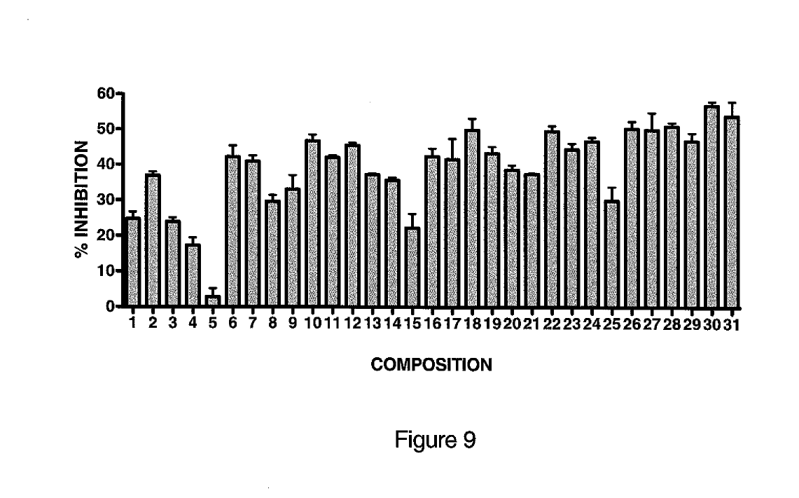

In this study, the compositions and α-synuclein were prepared as outlined above. The Congo red spectrophotometric assay was used to measure the disaggregation of α-synuclein fibrils. The results of this study are summarized in Table 8 and the α-synuclein: composition ratio of 1:1 shown graphically in FIG. 9. Compositions 1-5 (one component compositions) displayed variable inhibition rates ranging from 3-37%. Again, no correlation was observed regarding increased efficacies in the disassembly/disruption of α-synuclein amyloid fibrils with increasing number of components in the composition. Unexpectedly, some of the dual component compositions (compositions 9, 12, 14 and 15) displayed better than expected results when compared to a total based on addition of single component inhibition rates (shown in table 9 for 1:1 ratio)

TABLE 8
% inhibition of disaggregation of α-synuclein fibrils
measured using Congo Red assay

| Name | Average | Std. dev | % Inhibition | % Std. dev |
|---|---|---|---|---|
| α-syn only | 229049 | 12653 | 0.0 | 5.52 |
| 1 1:1 | 172474 | 4542 | 24.7 | 1.98 |
| 1 1:0.1 | 202284 | 1817 | 11.7 | 0.79 |
| 1 1:0.01 | 243514 | 11267 | −6.3 | 4.92 |
| 2 1:1 | 144493 | 2486 | 36.9 | 1.09 |
| 2 1:0.1 | 193969 | 7330 | 15.3 | 3.20 |
| 2 1:0.01 | 230978 | 8842 | −0.8 | 3.86 |
| 3 1:1 | 174361 | 2646 | 23.9 | 1.16 |
| 3 1:0.1 | 226323 | 7163 | 1.2 | 3.13 |
| 3 1:0.01 | 242387 | 11759 | −5.8 | 5.13 |
| 4 1:1 | 189583 | 5070 | 17.2 | 2.21 |
| 4 1:0.1 | 224137 | 8930 | 2.1 | 3.90 |
| 4 1:0.01 | 240133 | 4331 | −4.8 | 1.89 |
| 5 1:1 | 222697 | 5457 | 2.8 | 2.38 |
| 5 1:0.1 | 223416 | 5959 | 2.5 | 2.60 |
| 5 1:0.01 | 227107 | 4402 | 0.8 | 1.92 |
| 6 1:1 | 132408 | 7288 | 42.2 | 3.18 |
| 6 1:0.1 | 187470 | 4623 | 18.2 | 2.02 |
| 6 1:0.01 | 242183 | 3553 | −5.7 | 1.55 |
| 7 1:1 | 135282 | 3799 | 40.9 | 1.66 |
| 7 1:0.1 | 203882 | 6310 | 11.0 | 2.75 |
| 7 1:0.01 | 236799 | 5103 | −3.4 | 2.23 |
| 8 1:1 | 161160 | 4189 | 29.6 | 1.83 |
| 8 1:0.1 | 208189 | 6439 | 9.1 | 2.81 |
| 8 1:0.01 | 238947 | 6483 | −4.3 | 2.83 |
| 9 1:1 | 153155 | 9004 | 33.1 | 3.93 |
| 9 1:0.1 | 191879 | 5382 | 16.2 | 2.35 |
| 9 1:0.01 | 244161 | 3830 | −6.6 | 1.67 |
| 10 1:1 | 121811 | 3907 | 46.8 | 1.71 |
| 10 1:0.1 | 183652 | 8550 | 19.8 | 3.73 |
| 10 1:0.01 | 230965 | 3953 | −0.8 | 1.73 |
| 11 1:1 | 132624 | 1436 | 42.1 | 0.63 |
| 11 1:0.1 | 188850 | 6733 | 17.6 | 2.94 |
| 11 1:0.01 | 240193 | 4790 | −4.9 | 2.09 |
| 12 1:1 | 124848 | 1773 | 45.5 | 0.77 |
| 12 1:0.1 | 182126 | 4575 | 20.5 | 2.00 |
| 12 1:0.01 | 230654 | 804 | −0.7 | 0.35 |
| 13 1:1 | 143606 | 799 | 37.3 | 0.35 |
| 13 1:0.1 | 213624 | 3999 | 6.7 | 1.75 |
| 13 1:0.01 | 233911 | 4599 | −2.1 | 2.01 |
| 14 1:1 | 147340 | 1788 | 35.7 | 0.78 |
| 14 1:0.1 | 213678 | 3694 | 6.7 | 1.61 |
| 14 1:0.01 | 244241 | 3225 | −6.6 | 1.41 |
| 15 1:1 | 177927 | 9070 | 22.3 | 3.96 |
| 15 1:0.1 | 220568 | 4764 | 3.7 | 2.08 |
| 15 1:0.01 | 227665 | 15106 | 0.6 | 6.59 |
| 16 1:1 | 131714 | 5195 | 42.5 | 2.27 |
| 16 1:0.1 | 204025 | 4275 | 10.9 | 1.87 |
| 16 1:0.01 | 234368 | 2574 | −2.3 | 1.12 |
| 17 1:1 | 133646 | 13444 | 41.7 | 5.87 |
| 17 1:0.1 | 203832 | 12250 | 11.0 | 5.35 |
| 17 1:0.01 | 226640 | 6854 | 1.1 | 2.99 |
| 18 1:1 | 114231 | 7342 | 50.1 | 3.21 |
| 18 1:0.1 | 207017 | 4562 | 9.6 | 1.99 |
| 18 1:0.01 | 216415 | 2907 | 5.5 | 1.27 |
| 19 1:1 | 129565 | 4382 | 43.4 | 1.91 |
| 19 1:0.1 | 236113 | 5387 | −3.1 | 2.35 |
| 19 1:0.01 | 234507 | 8806 | −2.4 | 3.84 |
| 20 1:1 | 140164 | 2954 | 38.8 | 1.29 |
| 20 1:0.1 | 240695 | 3344 | −5.1 | 1.46 |
| 20 1:0.01 | 231763 | 11643 | −1.2 | 5.08 |
| 21 1:1 | 143029 | 385 | 37.6 | 0.17 |
| 21 1:0.1 | 216620 | 1850 | 5.4 | 0.81 |
| 21 1:0.01 | 225744 | 3455 | 1.4 | 1.51 |
| 22 1:1 | 115307 | 3647 | 49.7 | 1.59 |
| 22 1:0.1 | 216550 | 8559 | 5.5 | 3.74 |
| 22 1:0.01 | 248651 | 5594 | −8.6 | 2.44 |
| 23 1:1 | 126795 | 3777 | 44.6 | 1.65 |
| 23 1:0.1 | 209296 | 4493 | 8.6 | 1.96 |
| 23 1:0.01 | 220732 | 10958 | 3.6 | 4.78 |
| 24 1:1 | 121693 | 2592 | 46.9 | 1.13 |
| 24 1:0.1 | 213819 | 4692 | 6.6 | 2.05 |
| 24 1:0.01 | 241607 | 1145 | −5.5 | 0.50 |
| 25 1:1 | 160404 | 9069 | 30.0 | 3.96 |
| 25 1:0.1 | 240688 | 2170 | −5.1 | 0.95 |
| 25 1:0.01 | 224004 | 17546 | 2.2 | 7.66 |
| 26 1:1 | 113314 | 4870 | 50.5 | 2.13 |
| 26 1:0.1 | 203361 | 4615 | 11.2 | 2.02 |
| 26 1:0.01 | 223242 | 4038 | 2.5 | 1.76 |
| 27 1:1 | 114004 | 11266 | 50.2 | 4.92 |
| 27 1:0.1 | 197650 | 3347 | 13.7 | 1.46 |
| 27 1:0.01 | 219165 | 9889 | 4.3 | 4.32 |
| 28 1:1 | 111838 | 2603 | 51.2 | 1.14 |
| 28 1:0.1 | 204263 | 5520 | 10.8 | 2.41 |
| 28 1:0.01 | 237039 | 5283 | −3.5 | 2.31 |
| 29 1:1 | 121192 | 5167 | 47.1 | 2.26 |
| 29 1:0.1 | 209455 | 5034 | 8.6 | 2.20 |
| 29 1:0.01 | 228079 | 9503 | 0.4 | 4.15 |
| 30 1:1 | 97697 | 2594 | 57.3 | 1.13 |
| 30 1:0.1 | 198667 | 10772 | 13.3 | 4.70 |
| 30 1:0.01 | 224634 | 11425 | 1.9 | 4.99 |
| 31 1:1 | 104662 | 9411 | 54.3 | 4.11 |
| 31 1:0.1 | 187628 | 10309 | 18.1 | 4.50 |
| 31 1:0.01 | 200074 | 9999 | 12.7 | 4.37 |

TABLE 9
Comparison of expected and observed % inhibition of
disaggregation of α- synuclein fibrils measured using
Congo Red assay at 1:1.

| No. | Composition | Expected | Observed |
|---|---|---|---|
| 1 | 703 | — | 25 |
| 2 | GSE | — | 37 |
| 3 | GTE | — | 24 |
| 4 | GBE | — | 17 |
| 5 | CUR | — | 3 |
| 6 | 703 + GSE | 62 | 42 |
| 7 | 703 + GTE | 49 | 41 |
| 8 | 703 + GBE | 42 | 30 |
| 9 | 703 + CUR | 28 | 33 |
| 10 | GSE + GTE | 61 | 47 |
| 11 | GSE + GBE | 54 | 42 |
| 12 | GSE + CUR | 40 | 46 |
| 13 | GTE + GBE | 41 | 37 |
| 14 | GTE + CUR | 28 | 36 |
| 15 | GBE + CUR | 20 | 22 |
| 16 | 703 + GSE + GTE | 86 | 43 |
| 17 | 703 + GSE + GBE | 79 | 42 |
| 18 | 703 + GSE + CUR | 65 | 50 |
| 19 | 703 + GTE + GBE | 66 | 43 |
| 20 | 703 + GTE + CUR | 52 | 39 |
| 21 | 703 + GBE + CUR | 45 | 38 |
| 22 | GSE + GTE + GBE | 78 | 50 |
| 23 | GSE + GTE + CUR | 64 | 45 |
| 24 | GSE + GBE + CUR | 57 | 47 |
| 25 | GTE + GBE + CUR | 44 | 30 |
| 26 | 703 + GSE + GBE + CUR | 82 | 51 |
| 27 | 703 + GSE + GTE + GBE | 103 | 50 |
| 28 | 703 + GSE + GTE + CUR | 89 | 51 |
| 29 | 703 + GTE + GBE + CUR | 69 | 47 |
| 30 | GSE + GTE + GBE + CUR | 81 | 57 |
| 31 | 703 + GSE + GTE + GBE + | 106 | 54 |
| | CUR | | |

Again one might expect that compositions comprising more than one component would show at least additive effects in terms of overall efficacy of the composition in its ability to cause disaggregation of α-synuclein fibrils. However, this was not generally the case. For the results measured by Congo Red and as shown in FIG. 9 and table 9, for example, for twenty two of the thirty one compositions, the expected results far exceeded the observed results and it was therefore surprising that in only four cases (compositions 9, 12, 14 and 15) the actual observed results exceeded the expected results.

We claim:

1. A method for the treatment of amyloidosis in a mammal in need thereof comprising administration of a therapeutically effective amount of a composition comprising at least two of the substances selected from the group consisting of Uncaria tomentosa extract, Gingko Biloba, Green Tea Extract, Grape Seed Extract and Curcumin.

2. The method of claim 1, wherein the composition comprises Uncaria tomentosa extract and curcumin.

3. The method of claim 1, wherein the composition comprises Grape Seed Extract and Gingko Biloba.

4. The method of claim 1, wherein the composition comprises Grape Seed Extract and Curcumin.

5. The method of claim 1, wherein the composition comprises Green Tea Extract and Gingko Biloba.

6. The method of claim 1, wherein the composition comprises Green Tea Extract and curcumin.

7. The method of claim 1, wherein the composition comprises Gingko Biloba and curcumin.

8. The method of claim 1, wherein the amyloidosis is Alzheimer's Disease.

9. The method of claim 1, wherein the amyloidosis is Parkinson's Disease.

10. A method of treating the formation, deposition, accumulation, or persistence of amyloid fibrils, comprising treating the fibrils with an effective amount of a composition comprising at least two of the substances selected from the group consisting of Uncaria tomentosa extract, Gingko Biloba, Green Tea Extract, Grape Seed Extract and Curcumin.

11. The method of claim 10, wherein the composition comprises Uncaria tomentosa extract and curcumin.

12. The method of claim 10, wherein the composition comprises Grape Seed Extract and Gingko Biloba.

13. The method of claim 10, wherein the composition comprises Grape Seed Extract and Curcumin.

14. The method of claim 10, wherein the composition comprises Green Tea Extract and Gingko Biloba.

15. The method of claim 10, wherein the composition comprises Green Tea Extract and curcumin.

16. The method of claim 10, wherein the composition comprises Gingko Biloba and curcumin.

17. A method of treating the formation, deposition, accumulation, or persistence of α-synuclein fibrils, comprising treating the fibrils with an effective amount of a composition comprising at least two of the substances selected from the group consisting of Uncaria tomentosa extract, Gingko Biloba, Green Tea Extract, Grape Seed Extract and Curcumin.

18. The method of claim 17, wherein the composition comprises Uncaria tomentosa extract and curcumin.

19. The method of claim 17, wherein the composition comprises Grape Seed Extract and Gingko Biloba.

20. The method of claim 17, wherein the composition comprises Grape Seed Extract and Curcumin.

21. The method of claim 17, wherein the composition comprises Green Tea Extract and Gingko Biloba.

22. The method of claim 17, wherein the composition comprises Green Tea Extract and curcumin.

23. The method of claim 17, wherein the composition comprises Gingko Biloba and curcumin.
